SEQUENCE LISTING (11 sequences)

SEQ ID NO: 1 - protein, 652 aa, Artificial Sequence
  Detoxified EPA protein comprising 4 optimized N-glycosylation sequences
GSGGGDQNATGSGGGKLAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKDNNNSTPTVISHRLHFPEGGSLAALTAHQACHLPLEAFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAKDQNRTKGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRVTILGWPLAERTVVIPSAIPTDPRNVGGDLDPSSIPDKEQAISALPDYASQPGKPPREDLKLGSGGGDQNAT

SEQ ID NO: 2 - protein, 5 aa, Artificial Sequence
  optimized N-glycosylation consensus sequence
  MISC_FEATURE (1)..(1): Xaa can be Asp or Glu
  MISC_FEATURE (2)..(2): Xaa can be any natural amino acid except Pro
  MISC_FEATURE (4)..(4): Xaa can be any natural amino acid except Pro
  MISC_FEATURE (5)..(5): Xaa can be Ser or Thr
XXNXX

SEQ ID NO: 3 - protein, 3 aa, Artificial Sequence
  N-glycosylation consensus sequence
  MOD_RES (2)..(2): Xaa can be any natural amino acid except Pro
  MOD_RES (3)..(3): Xaa can be Ser or Thr
NXX

SEQ ID NO: 4 - protein, 300 aa, Artificial Sequence
  FimH full length
MKRVITLFAVLLMGWSVNAWSFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

SEQ ID NO: 5 - protein, 186 aa, Artificial Sequence
  example FimHt sequence
FACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGLVIKAGSLIAVLILRQTNNYNSDDFQFVWNIYANNDVVVPTGGCDVSARDVTVTLPDYRGSVPIPLTVY

SEQ ID NO: 6 - protein, 300 aa, Artificial Sequence
  example full length FimH sequence
MKRVITLFAVLLMGWSVNAWSFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYRGSVPIPLTVYCAKSQNLGYYLSGTHADAGNSIFTNTASFSPAQGVGVQLTRNGTIIPANNTVSLGAVGTSAVSLGLTANYARTGGQVTAGNVQSIIGVTFVTQ

SEQ ID NO: 7 - protein, 279 aa, Artificial Sequence
  example FimH sequence with truncation at N- terminus
FACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGLVIKAGSLIAVLILRQTNNYNSDDFQFVWNIYANNDVVVPTGGCDVSARDVTVTLPDYRGSVPIPLTVYCAKSQNLGYYLSGTHADAGNSIFTNTASFSPAQGVGVQLTRNGTIIPTNNTVSLGAVGTSAVSLGLTANYARTGGQVTAGNVQSIIGVTFVYQ

SEQ ID NO: 8 - protein, 160 aa, Artificial Sequence
  example FimH-LD sequence
FACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGLVIKAGSLIAVLILRQTNNYNSDDFQFVWNIYANNDVVVPTGG

SEQ ID NO: 9 - protein, 159 aa, Artificial Sequence
  example FimH-LD sequence (FimH-LD 23-10)
FACKTANGTAIPIGGGSANVYVNLAPAVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTG

SEQ ID NO: 10 - protein, 300 aa, Artificial Sequence
  example FimH full length sequence (23-10)
MKRVITLFAVLLMGWSVNAWSFACKTANGTAIPIGGGSANVYVNLAPAVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

SEQ ID NO: 11 - protein, 279 aa, Artificial Sequence
  example FimH (23-10) sequence with truncation at N-terminus
FACKTANGTAIPIGGGSANVYVNLAPAVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

(19) United States
(12) Patent Application Publication
(10) Pub. No.: US 2019/0275134 A1
(43) Pub. Date: Sep. 12, 2019

(54) VACCINES AGAINST URINARY TRACT INFECTIONS

(71) Applicants: Janssen Pharmaceuticals, Inc, Titusville, NJ (US); Janssen Vaccines & Prevention B.V., Leiden (NL)

(72) Inventors: Jan Theunis Poolman, Haarlem (NL); Jeroen Geurtsen, Vleuten (NL); Kellen Cristhina Fae, Oegstgeest (NL)

(21) Appl. No.: 16/299,907

(22) Filed: Mar. 12, 2019

(30) Foreign Application Priority Data
Mar. 12, 2018 (EP) 18 161 252.4
Aug. 23, 2018 (EP) 18 190 402.0

(51) Int. Cl. A61K 39/108

(52) U.S. Cl. CPC A61K 39/0258; A61K 2039/55577

(57) ABSTRACT

Compositions and methods are described for vaccinating against E. coli urinary tract infections. The compositions contain a FimH polypeptide, one or more conjugates containing E. coli O-antigens polysaccharide covalently coupled to a carrier protein, and an adjuvant.

### CROSS-REFERENCE TO RELATED APPLICATIONS

This application claims priority under 35 U.S.C. § 119(b) to European Patent Application No. EP 18 161 252.4, filed Mar. 12, 2018, and European Patent Application No. EP 18 190 402.0 filed Aug. 23, 2018, the disclosures of which are incorporated herein by reference.

### REFERENCE TO SEQUENCE LISTING SUBMITTED ELECTRONICALLY

This application contains a sequence listing which is submitted electronically via EFS-Web as an ASCII formatted sequence listing with a file name of “688097-733US,” a creation date of “Mar. 12, 2019,” and having a size of “25 KB.” The sequence listing submitted via EFS-Web is part of the specification and is herein incorporated by reference in its entirety.

### FIELD OF THE INVENTION

This invention relates to compositions and methods for vaccination against urinary tract infections. In particular, embodiments of this invention relate to multivalent vaccines containing FimH polypeptide, conjugates of E. coli O-antigen polysaccharides covalently bound to carrier proteins, and an adjuvant, and uses of the vaccines to protect against urinary tract infections caused by E. coli.

### BACKGROUND OF THE INVENTION

Urinary tract infections (UTIs) are an important health care problem in young females and older adults. Uropathogenic E. coli (UPEC), a type of extra-intestinal pathogenic E. coli (ExPEC), is responsible for many of these infections. Today, symptomatic UTIs are primarily treated using antibiotics. Although first-line antibiotic treatment is effective in most cases, the rise in antibiotic-resistant strains causes this treatment method to become more prone to failure, which can result in more difficult to treat disease. In addition, E. coli is known to cause recurrent infections even in patients with a history of antibiotic treatment. Given these circumstances, the need for alternative treatment options, and more preferably for preventing UTIs, is apparent. A vaccine that effectively prevents E. coli UTIs is currently not available. The high degree of diversity among the UPEC population complicates vaccine design (Brumbaugh A R and Mobley H L T, 2012, Expert Rev Vaccines, 11: 663-676). In addition, the bladder is an immunotolerized immunological compartment, and induction of mucosal immunity through vaccination in general is a rather difficult task.

A phase 1b, first in human, clinical trial of a bio-conjugate vaccine against ExPEC, comprising four different E. coli O-antigens covalently bound to a carrier protein, demonstrated elicitation of functional opsonophagocytic antibodies against all vaccine serotypes in women with a history of recurrent UTIs (Huttner A, et al., 2017, Lancet Infect Dis, dx.doi.org/10.1016/S1473-3099(17)30108-1). As a secondary outcome, this study demonstrated partial protective effectiveness against recurrent UTI with high (≥10⁵ cfu/mL) bacterial load. The induction of functional antibodies in blood raises the expectation that such ExPEC conjugates might be able to prevent invasive disease including bacteremia, but that for vaccination aimed at preventing UTIs, the efficacy of the vaccine may need to be further improved.

The FimH adhesin protein has been shown to induce protection in various pre-clinical models against UTI (Langermann S, et al., 1997, Science, 276: 607-611; Langermann S, et al., 2000, J Infect Dis, 181: 774-778; O'Brien V P et al., 2016, Nat Microbiol, 2:16196). In 1999, Medimmune brought a FimH-containing subunit vaccine to phase II trials, but development of the vaccine was discontinued in 2003 for lack of efficacy in prevention of UTIs (see, e.g., Brumbaugh A R and Mobley H L T, supra). Nevertheless, the company Sequoia Sciences appears to be currently clinically developing a vaccine for recurrent UTIs, the vaccine consisting of the FimH protein combined with a new adjuvant formulation. The company reports that this vaccine was highly immunogenic and well-tolerated and may reduce the frequency of UTI, although safety and efficacy still need to be established (https://www.sequoiasciences.com/uti-vaccine-program).

As indicated above, there remains a need in the art for vaccines that can reduce the incidence of UTIs.

It is an object of the present invention to provide novel vaccine compositions that can induce broad protection against, and thereby contribute to reduce the incidence of, E. coli UTIs.

### BRIEF SUMMARY OF THE INVENTION

The invention provides vaccine combinations of FimH polypeptide with E. coli O-antigens conjugated to carrier protein, and adjuvant, for protection against E. coli UTIs. Such vaccine combinations provide a combination of different mechanisms of action, viz. induction of FimH-specific antibodies that inhibit bacterial adhesion to bladder epithelial cells and induction of O-antigen-specific opsonophagocytic antibodies that mediate bacterial killing. These combinations are thus expected to have combined effects over each of the individual antigens, i.e. at least additive and can give synergistic effects. An adjuvant (for instance, TLR4-agonist) is expected to increase the immune responses to at least FimH, likely also to the O-antigens, and may activate T cell responses with a predominant Th1-inflammatory function at the mucosal site.

Accordingly, in one general aspect, the invention relates to a vaccine combination, comprising (i) a FimH polypeptide, (ii) one or more conjugates comprising an E. coli O-antigen polysaccharide covalently coupled to a carrier protein, and (iii) an adjuvant. In one embodiment, the vaccine combination comprises a first composition comprising (i), a second composition comprising (ii) and a third composition comprising (iii). In another embodiment, the vaccine combination comprises a first composition comprising (i) and (ii) and a second composition comprising (iii). In another embodiment, the vaccine combination comprises a first composition comprising (i) and (iii) and a second composition comprising (ii). In another embodiment, the vaccine combination comprises a first composition comprising (i) and a second composition comprising (ii) and (iii). In a preferred embodiment, the vaccine combination comprises a composition comprising (i), (ii) and (iii). In another preferred embodiment, the vaccine combination comprises a first composition and a second composition as above, or a first, second and third composition as above, wherein the first and second composition, or the first, second and third composition, are for administration to the subject within a time frame and at a location that allows draining of the vaccine combination components to the same lymph node.

In certain embodiments, the one or more conjugates comprise E. coli O25B antigen polysaccharide.

In certain embodiments, the one or more conjugates comprise E. coli O25B antigen polysaccharide, E. coli O1A antigen polysaccharide, E. coli O2 antigen polysaccharide, and E. coli O6A antigen polysaccharide.

In certain embodiments, the one or more conjugates comprise E. coli O25B antigen polysaccharide, E. coli O1A antigen polysaccharide, E. coli O2 antigen polysaccharide, E. coli O6A antigen polysaccharide, and 1 to 20, e.g. 1, 2, 3, 4, 5, 6, 7, 8, 9, 10, 11, 12, 13, 14, 15, 16, 17, 18, 19, or 20 additional E. coli O-antigen polysaccharides. In certain non-limiting embodiments, one or more of the 1 to 20 additional E. coli O-antigen polysaccharides comprise one or more of O4, O7, O9, O11, O12, O22, O75, O8, O15, O16, or O18 antigen polysaccharides.

In certain embodiments, the carrier protein is detoxified exotoxin A of Pseudomonas aeruginosa (EPA). In a preferred embodiment, the carrier protein comprises the amino acid sequence of SEQ ID NO: 1.

In certain embodiments, the FimH polypeptide comprises truncated FimH. In certain embodiments, the FimH polypeptide comprises the amino acid sequence of SEQ ID NO: 5. In certain embodiments, the FimH polypeptide comprises the amino acid sequence of SEQ ID NO: 8. In certain embodiments, the FimH polypeptide comprises the amino acid sequence of SEQ ID NO: 9. In certain embodiments the FimH polypeptide comprises FimH in a high affinity conformation. In certain embodiments, the FimH polypeptide comprises FimH in a low affinity conformation, e.g. a FimH variant with a mutation R60P (wherein the numbering corresponds to the amino acid numbering in SEQ ID NO: 9). In certain embodiments, the FimH polypeptide is complexed with FimC (referred to as FimCH).

In certain preferred embodiments, the adjuvant comprises a saponin-based adjuvant, such as an adjuvant containing the water-extractable fraction of saponins from Quillaja saponaria. In certain embodiments, the adjuvant comprises QS21.

In certain preferred embodiments, the adjuvant comprises liposomes, in certain embodiments such liposomes comprise saponins such as QS21.

In certain preferred embodiments, the adjuvant comprises a TLR4 agonist. In certain embodiments, the adjuvant comprises a lipid A analog. In certain embodiments thereof, the TLR4 agonist comprises MPL, 3D-MPL, RC529, GLA, SLA, E6020, PET-lipid A, PHAD, 3D-PHAD, 3D-(6-acyl)-PHAD, ONO4007, or OM-174.

It is also an aspect of the invention to provide a method for vaccinating against a UTI caused by E. coli in a subject in need thereof, comprising administering to the subject a vaccine combination of the invention. The FimH polypeptide, the at least one E. coli O-antigen polysaccharide covalently coupled to a carrier protein, and the adjuvant can be administered in one composition, or they can be administered in combination from multiple compositions.

In a further aspect, the invention provides a method for making vaccine combinations of the invention, the method comprises combining (i) (the FimH polypeptide), (ii) (the at least one E. coli O-antigen polysaccharide covalently coupled to a carrier protein), and (iii) (the adjuvant), to thereby obtain the vaccine combination. In certain embodiments, the components of the vaccine combination are present in a kit. In certain embodiments, the method for making a vaccine combination of the invention comprises combining (i), (ii), and a pharmaceutically acceptable carrier in a first composition, preparing a second composition comprising (iii), and combining the first composition with the second composition to obtain the vaccine combination. In one embodiment, the first composition and the second composition are combined into a mixed composition shortly before administration to the subject. In other embodiments, the vaccine combination is administered by multiple compositions that each comprise part of the components of the total vaccine combination that comprises (i) a FimH polypeptide, (ii) one or more conjugates comprising an E. coli O-antigen polysaccharide covalently coupled to a carrier protein, and (iii) an adjuvant, e.g. wherein a first of the multiple compositions comprises (i), a second of the multiple compositions comprises (ii), and a third of the multiple compositions comprises (iii); or wherein a first of the multiple compositions comprises (i) and (iii), and a second of the multiple compositions comprises (ii); or wherein a first of the multiple compositions comprises (i) and (ii), and a second of the multiple compositions comprises (iii); or wherein a first of the multiple compositions comprises (ii) and (iii), and a second of the multiple compositions comprises (i); wherein the multiple compositions are administered to a subject within a time frame and at a location that allows draining of the vaccine combination components to the same lymph node.

The invention also relates to use of a vaccine combination according to the invention for the manufacture of a vaccine or medicament for preventing UTI, or for reducing the chance of suffering from or for reducing the severity of one or more symptoms associated with UTI in a subject in need thereof. The invention also relates to a vaccine combination according to the invention for use in the prevention of UTI or for reduction of the chance of suffering from or for reduction of the severity of one or more symptoms associated with UTI in a subject in need thereof.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

FIG. 1 shows the inhibition of bacterial adhesion to bladder epithelial cells mediated by FimH-specific antibodies. Data shows % of bacterial (E. coli J96) adhesion to bladder epithelial cells (5637 cell line) without serum (dotted line) and inhibition of adhesion mediated by serum samples (mean±SD) from rats pre-vaccination and post-vaccination with 2 different variants of FimH (FimH_{LD} 23-10 and FimH_{LD} 23-10 R60P).

FIG. 2 shows the experimental design of an immunogenicity and efficacy study (see Example 4 for details). *: time point for blood draw and serum antibodies measurements. ^{#}: time point for evaluation of functionality of antibodies, T and B cell responses. ^{&}Bladder and kidney CFU are determined at 4 hours and at 6 days post infection.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning commonly understood to one of ordinary skill in the art to which this invention pertains. Otherwise, certain terms cited herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “pharmaceutically acceptable carrier” refers to a non-toxic material that does not interfere with the effectiveness of a composition according to the invention or the biological activity of a composition according to the invention. A “pharmaceutically acceptable carrier” can include any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the pharmaceutically acceptable carrier will depend on the route of administration for a particular application. According to particular embodiments, in view of the present disclosure, any pharmaceutically acceptable carrier suitable for use in a vaccine can be used in the invention. Suitable excipients include but are not limited to sterile water, saline, dextrose, glycerol, ethanol, or the like and combinations thereof, as well as stabilizers, e.g. Human Serum Albumin (HSA) or other suitable proteins and reducing sugars.

As used herein, the term “effective amount” refers to an amount of an active ingredient or component that elicits the desired biological or medicinal response in a subject. An effective amount can be determined empirically and in a routine manner, in relation to the stated purpose. For example, in vitro assays can optionally be employed to help identify optimal dosage ranges.

As used herein, the term “in combination,” in the context of the administration of one or more O-antigens, FimH and adjuvant, or compositions comprising these components to a subject, does not restrict the order in which O-antigens, FimH and adjuvant or compositions comprising these are administered to a subject. For example, a first composition (e.g. comprising first components, e.g. conjugate of O-antigen and FimH) can be administered prior to (e.g., 5 minutes, 15 minutes, 30 minutes, 45 minutes, 1 hour, 2 hours, 4 hours, 6 hours, or 12 hours before), concomitantly with, or subsequent to (e.g., 5 minutes, 15 minutes, 30 minutes, 45 minutes, 1 hour, 2 hours, 4 hours, 6 hours, or 12 hours after) the administration of a second composition (e.g. comprising a second component, e.g. adjuvant) to a subject. In certain embodiments, the E. coli O-antigen and the FimH polypeptide are present in a first composition, the adjuvant is present in a second composition, and the first and second compositions are combined shortly before administration, in a mix-and-shoot application.

In certain embodiments, the vaccine combination is administered by multiple compositions that each comprise a part of the total vaccine combination that comprises (i) a FimH polypeptide, (ii) one or more conjugates comprising an E. coli O-antigen polysaccharide covalently coupled to a carrier protein, and (iii) an adjuvant, e.g. wherein a first of the multiple compositions comprises (i), a second of the multiple compositions comprises (ii), and a third of the multiple compositions comprises (iii); or wherein a first of the multiple compositions comprises (i) and (iii), and a second of the multiple compositions comprises (ii); or wherein a first of the multiple compositions comprises (i) and (ii), and a second of the multiple compositions comprises (iii); or wherein a first of the multiple compositions comprises (ii) and (iii), and a second of the multiple compositions comprises (i); wherein in preferred embodiments thereof the multiple compositions are administered to the subject in the same limb at a short distance of each other, e.g. within 30 cm, 20 cm, within 10 cm, within 5 cm, within 2 cm of each other, and within a few days of each other, e.g. within 72 hours, 48 hours, 24 hours, 8 hours, preferably within 2 hours, within 1 hour, within 30 minutes, within 10 minutes, preferably within 5 minutes, within 2 minutes, preferably co-administered essentially simultaneously. This will enable the vaccine components to drain to the same lymph node, which will ensure a maximal benefit from the adjuvant, even without physical combination or mix-and-shoot. This also works when adjuvant is provided within a few days of antigens. In certain embodiments therefore, the vaccine combination is administered to a subject by multiple compositions within a time frame and at a location that allows draining of the vaccine combination components to the same lymph node.

As used herein, the term “extra-intestinal pathogenic E. coli” or “ExPEC” refers to genetically related pathogenic E. coli strains that commonly invade, colonize, and induce disease in bodily sites outside of the gastrointestinal tract. ExPEC bacteria include uropathogenic (UPEC) E. coli, newborn meningitic (NMEC) E. coli, septicaemia associated (SePEC) E. coli, adherent invasive (AIEC) E. coli, and avian pathogenic (APEC) E. coli. Diseases associated with ExPEC or ExPEC infections include, but are not limited to, urinary tract infection, surgical-site infection, bacteremia, abdominal or pelvic infection, such as intra-abdominal infections, pneumonia, nosocomial pneumonia, osteomyelitis, cellulitis, pyelonephritis, wound infection, meningitis, neonatal meningitis, peritonitis, cholangitis, soft-tissue infections, pyomyositis, septic arthritis, and sepsis.

As used herein, the term “urinary tract infection” or “UTI” refers to a bacterial infection that affects parts of the body that produce and/or carry urine, i.e. the urinary tract, e.g. kidney, ureter, bladder and/or urethra. When it affects the lower urinary tract it is also known as a bladder infection (cystitis), and when it infects the upper urinary tract it is also known as kidney infection (pyelonephritis). Symptoms from a lower UTI can include pain with urination, frequent urination, and feeling the need to urinate despite having an empty bladder, while symptoms of kidney infection can include fever and flank pain usually in combination with the symptoms of a lower UTI. UTI can also lead to life-threatening invasive E. coli disease, e.g. bacteremia, sepsis, or urosepsis. The most common cause of UTI is E. coli. Risk factors include female anatomy, sexual intercourse, diabetes, obesity and family history. UTIs are more common in women than in men, and occur frequently between the ages of 16 and 35 years. UTIs also occur frequently in elderly men and women. Recurrences of UTI are common, and “recurrent UTI” or “rUTI” refers to at least two infections in six months or at least three infections in one year. Catheterization is also a risk factor for UTI (CAUTI: Catheter-Associated-UTI) and a major contributor to the totality of health-care-associated infections (HAI).

As used herein, “subject” or “patient” means any animal, preferably a mammal, most preferably a human, who will be or has been vaccinated by a method or composition according to an embodiment of the invention. The term “mammal” as used herein, encompasses any mammal. Examples of mammals include, but are not limited to, cows, horses, sheep, pigs, cats, dogs, mice, rats, rabbits, guinea pigs, monkeys, humans, etc., most preferably a human. In certain embodiments, a subject is a human adult. As used herein, the term “human adult” refers to a human that is 18 years or older.

As used herein, an “immunological response” or “immune response” to an antigen or composition refers to the development in a subject of a humoral and/or a cellular immune response to the antigen or an antigen present in the composition.

### Epidemiology

Studies on the distribution of E. coli serotypes causing UTIs identified serotypes O1, O2, O6, and O25 amongst the most prevalent E. coli serotypes found in target populations (see, e.g., the disclosure of WO 2017/035181, which is incorporated by reference herein). It was also described that, for an O-antigen serotype that is composed of distinct, yet structurally and antigenically related subtypes, one subtype can be more prevalent among clinical isolates than others. For example, O1A, O6A and O25B antigens were determined to be the more frequent subtypes among the recently analyzed clinical strains or isolates for O1, O6 and O25 serotypes, respectively. See related disclosure on epidemiology studies in WO 2015/124769, the disclosure of which is incorporated by reference herein.

Compositions Comprising E. coli O-Antigen Conjugates, FimH and Adjuvant

In one general aspect, the invention relates to multivalent vaccines comprising one or more E. coli O-antigen conjugates, FimH polypeptide, and adjuvant.

E. coli O-Antigens and Conjugates

The O-antigen serotype is based on the O polysaccharide antigen, the surface polysaccharide part of the lipopolysaccharide (LPS) in a Gram-negative bacterium. More than 180 E. coli O-antigens have been reported (Stenutz et al., 2006, FEMS Microbial Rev, 30: 382-403). As used herein, the terms “O polysaccharide,” “O-antigen,” “O-antigen polysaccharide,” “O-polysaccharide antigen” and the abbreviation “OPS” all refer to the O-antigen of Gram-negative bacteria, which is the outer membrane portion of the LPS and is specific for each serotype or sero(sub)type of the Gram-negative bacteria, the Gram-negative bacteria here being E. coli. The O-antigen usually contains a polymer of repeating units (RUs), the RU typically consisting of two to seven sugar residues. As used herein, the RU is set equal to the biological repeat unit (BRU). The BRU describes the RU of an O-antigen as it is synthesized in vivo.

As used herein, the terms “conjugate” and “glycoconjugate” refer to a conjugation product containing an E. coli O-antigen covalently bound to a carrier protein. The conjugate can be a bioconjugate, which is a conjugation product prepared in a host cell, wherein the host cell machinery produces the O-antigen and the carrier protein and links the O-antigen to the carrier protein enzymatically, e.g., via N-linkages. In preferred embodiments, the conjugate is a bioconjugate, which can be prepared according to methods for instance described in WO 2015/124769. The conjugate can also be prepared by other means, for example, by chemical linkage of the purified carrier protein and O-antigen or O-antigen containing structures. In the case of chemical conjugations, the starting polysaccharide can be purified from bacteria or the polysaccharide can be synthesized in vitro chemically and/or enzymatically, and then the polysaccharide can be conjugated to carrier protein chemically or enzymatically.

In certain embodiments, the O-antigen conjugates contain O-antigen serotypes found predominantly among E. coli clinical isolates, which can be used to provide active immunization for the prevention of disease caused by E. coli having the O-antigen serotypes contained in the vaccine. Preferably, the compositions according to embodiments of the invention comprise conjugates of more than one E. coli O-antigen, which are prevalent among E. coli clinical isolates. Examples of such O-antigens include, but are not limited to, E. coli O1, O2, O4, O6, O7, O8, O9, O11, O12, O15, O16, O17, O18, O21, O22, O25, O44, O73, O75, O77, O101, and O153 antigens. Depending on the need, the composition can include more than one E. coli O-antigen, such as 2, 3, 4, 5, 6, 7, 8, 9, 10, 11, 12, 13, 14, 15, 16, 17, 18, 19, 20, or more E. coli O-antigens, to provide immune protection against multiple E. coli serotypes. In a preferred embodiment, the compositions at least comprise a conjugate with O-antigen from E. coli O25B serotype. In a preferred embodiment, additional E. coli O-antigens are selected from the group consisting of E. coli O1, O2 and O6 antigens. More preferably, the composition comprises conjugates of E. coli O-antigen from E. coli O25B, O1A, O2 and O6A. In certain embodiments, the compositions in addition to O25B, O1A, O2 and O6A O-antigen conjugates further comprise 1-16, e.g. 1-10, additional conjugates having O-antigens from additional E. coli serotypes. In one exemplary and non-limiting embodiment, such additional serotypes comprise one or more from O4, O7, O9, O11, O12, O22, O75, O8, O18, O15, and O16. Conjugates comprising O-antigens of other E. coli serotypes may be added or used instead of the ones mentioned above, e.g. based upon epidemiologic studies in the target population.

Cryz et al, 1995, Vaccine 13: 449-453, disclosed a 12-valent composition comprising O-antigens of E. coli LPS serotypes O1, O2, O4, O6, O7, O8, O12, O15, O16, O18, O25(A) and O75. Fattom et al, 1999, supra, disclosed a 12-valent composition comprising O-antigens of E. coli LPS serotypes O1, O4, O6, O7, O8, O11, O15, O16, O18, O22, O25 (likely O25A) and O75.

As used herein an “E. coli O25B antigen” refers to an O-antigen specific to the E. coli O25B serotype. In one embodiment, an E. coli O25B antigen comprises the structure of Formula O25B′:

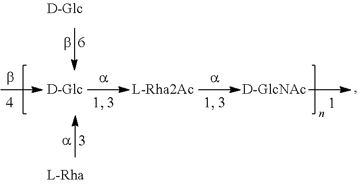

wherein the n in Formula O25B′ is an integer of 1 to 30, 1 to 20, 1 to 15, 1 to 10, 1 to 5, 10 to 30, 15 to 30, 20 to 30, 25 to 30, 5 to 25, 10 to 25, 15 to 25, 20 to 25, 10 to 20, or 15 to 20. In one embodiment of the invention, the n in Formula O25B′ is an integer of 10-20.

Preferably, a population of E. coli O25B antigens having the structure of Formula O25B′, is used in compositions and methods according to embodiments of the invention, wherein the n of at least 80% of the E. coli O25B antigens in the population is an integer of 1 to 30, 1 to 20, 1 to 15, 1 to 10, 1 to 5, 10 to 30, 15 to 30, 20 to 30, 25 to 30, 5 to 25, 10 to 25, 15 to 25, 20 to 25, 10 to 20, or 15 to 20. In one embodiment of the invention, the n of at least 80% of the E. coli O25B antigens in the population is an integer of 10-20.

As used herein, an “E. coli O1 antigen” refers to an O-antigen specific to the E. coli O1 serotype. In one embodiment, an E. coli O1 antigen is an E. coli O1A antigen.

As used herein, an “E. coli O1A antigen” refers to an O-antigen specific to the E. coli O1A serotype. In one embodiment, an E. coli O1A antigen comprises the structure of Formula O1A′:

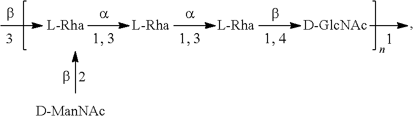

wherein the n in Formula O1A′ is an integer of 1 to 30, 1 to 20, 1 to 15, 1 to 10, 1 to 5, 10 to 30, 15 to 30, 20 to 30, 25 to 30, 5 to 25, 10 to 25, 15 to 25, 20 to 25, 10 to 20, or 15 to 20. In one embodiment, the n in Formula O1A′ is an integer of 7-15.

Preferably, a population of E. coli O1A antigens having the structure of Formula 01A′, is used in compositions and methods according to embodiments of the invention, wherein the n of at least 80% of the E. coli O1A antigens in the population is of 1 to 30, 1 to 20, 1 to 15, 1 to 10, 1 to 5, 10 to 30, 15 to 30, 20 to 30, 25 to 30, 5 to 25, 10 to 25, 15 to 25, 20 to 25, 10 to 20, or 15 to 20. In one embodiment, then of at least 80% of the E. coli O1A antigens in the population is an integer of 5-15.

As used herein, an “E. coli O2 antigen” refers to an O-antigen specific to the E. coli O2 serotype. In one embodiment, an E. coli O2 antigen comprises the structure of Formula O2′:

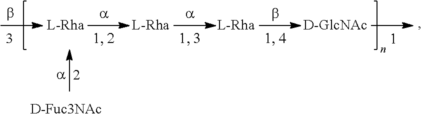

wherein the n in Formula O2′ is an integer of 1 to 30, 1 to 20, 1 to 15, 1 to 10, 1 to 5, 10 to 30, 15 to 30, 20 to 30, 25 to 30, 5 to 25, 10 to 25, 15 to 25, 20 to 25, 10 to 20, or 15 to 20. In one embodiment, then in Formula O2′ is an integer of 8-16.

Preferably, a population of E. coli O2 antigens having the structure of Formula O2′, is used in compositions and methods according to embodiments of the invention, wherein the n of at least 80% of the E. coli O2 antigens in the population is of 1 to 30, 1 to 20, 1 to 15, 1 to 10, 1 to 5, 10 to 30, 15 to 30, 20 to 30, 25 to 30, 5 to 25, 10 to 25, 15 to 25, 20 to 25, 10 to 20, or 15 to 20. In one embodiment, the n of at least 80% of the E. coli O2 antigens in the population is an integer of 5-20.

As used herein, an “E. coli O6 antigen” refers to an O-antigen specific to the E. coli O6 serotype. In one embodiment, an E. coli O6 antigen is an E. coli O6A.

As used herein, an “E. coli O6A antigen,” also referred to as “E. coli O6K2 antigen” or “E. coli O6Glc antigen,” refers to an O-antigen specific to the E. coli O6A serotype. In one embodiment, an E. coli O6A antigen comprises the structure of Formula O6A′:

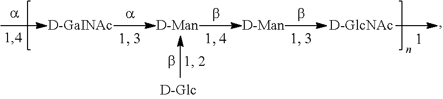

wherein the β1, 2 linkage is also named β2 linkage, the n in Formula O6A′ is an integer of 1 to 30, 1 to 20, 1 to 15, 1 to 10, 1 to 5, 10 to 30, 15 to 30, 20 to 30, 25 to 30, 5 to 25, 10 to 25, 15 to 25, 20 to 25, 10 to 20, or 15 to 20. In one embodiment, the n in Formula O6A′ is an integer of 8-18.

Preferably, a population of E. coli O6A antigens having the structure of Formula O6A′, is used in compositions and methods according to embodiments of the invention, wherein the n of at least 80% of the E. coli O6A antigens in the population is of 1 to 30, 1 to 20, 1 to 15, 1 to 10, 1 to 5, 10 to 30, 15 to 30, 20 to 30, 25 to 30, 5 to 25, 10 to 25, 15 to 25, 20 to 25, 10 to 20, or 15 to 20. In one embodiment, the n of at least 80% of the E. coli O6A antigens in the population is an integer of 5-20.

In a preferred embodiment, a composition of the invention comprises E. coli O25B antigens having the structure of formula O25B′, wherein the n of at least 80% of the E. coli O25B antigens in the population is an integer of 10-20; E. coli O1A antigens having the structure of formula O1A′, wherein the n of at least 80% of the E. coli O1A antigens in the population is an integer of 5-15; E. coli O2 antigens having the structure of formula O2′, wherein the n of at least 80% of the E. coli O2 antigens in the population is an integer of 5-20; and E. coli O6A antigens having the structure of formula O6A′, wherein the n of at least 80% of the E. coli O6A antigens in the population is an integer of 5-20, wherein each of the O-antigens is covalently bound to an EPA carrier protein having the amino acid sequence of SEQ ID NO:1.

An E. coli O-antigen useful in the invention can be produced by methods known in the art in view of the present disclosure. For example, they can be produced from a cell, preferably a recombinant cell that is optimized for the biosynthesis of the O-antigen. See, e.g., relevant disclosure on the nucleic acids, proteins, host cells, production methods, etc., for E. coli O-antigen biosynthesis in WO 2006/119987, WO 2009/104074, WO 2015/124769, Ihssen et al., 2010, Microbial Cell Factories, 9:61, the disclosures of which are incorporated by reference herein. E. coli O-antigens useful in the invention can also be produced by traditional extraction methods including those using, e.g., trichloroacetic acid, aqueous butanol, triton/Mg⁺², cold ethanol or water at 100° C., phenol, chloroform, petroleum-ether or methanol (see, e.g., Apicella et al., 1994, Methods Enzymol, 235:242-52). E. coli O-antigens useful in the invention can also be produced by in vitro chemical synthesis of polysaccharides using methods known in the art (see, e.g., Woodward et al., 2010, Nat Chem Biol, 6(6): 418-423).

The effective amount or dosage of a conjugate is defined based on the polysaccharide moiety in the conjugate. In compositions comprising more than one conjugate, the concentration of each conjugate can be about the same, or different conjugates can be present in different concentrations.

For compositions comprising conjugates of O25B antigen and conjugates of for instance O1A, O2 and O6A antigen, the concentration or dosage of the O1A, O2 and O6A antigen conjugates typically is between 20 and 100% of the O25B antigen conjugate. Some non-limiting examples of compositions comprising conjugates of O25B, O1A, O2 and O6A comprise these conjugates at a weight ratio (of the respective antigen polysaccharides) of 1:1:1:1, or 2:1:1:1, or 4:1:1:1, or 4:2:1:1.

Non-limiting exemplary dosages for a single administration to a subject are for instance between about 2 and 25 microgram (ug) per individual polysaccharide, for instance between about 4 and 16 ug per polysaccharide.

A typical volume for administration by injection to a human subject is between about 0.1-1.5, most typically about 0.5 mL.

In certain embodiments, the concentration of the O25B conjugate in the composition is between about 5 and about 50 microgram (ug)/mL, preferably between 8 and 32 ug/mL, e.g. 8, 12, 16, 20, 24, 28 or 32 ug/mL, more preferably between 8 and 16 ug/mL.

A non-limiting and exemplary administration dose for a composition wherein O25B would for example be present at 16 ug/mL and comprising conjugates of O25B, O1A, O2 and O6A at a weight ratio (of the respective antigen polysaccharides) of for example 2:1:1:1, would be 8:4:4:4 ug polysaccharide for O25B:O1A:O2:O6A conjugates. Another non-limiting and exemplary administration dose for a composition comprising wherein O25B would for example be present at 8 ug/mL and comprising conjugates of O25B:O1A:O2:O6A at a weight ratio of respective antigen polysaccharides of for example 1:1:1:1 would be 4:4:4:4 ug polysaccharide for the respective conjugates, etc. Such compositions and dosages have been described in more detail in WO 2017/035181, incorporated by reference, and have been tested in humans.

For compositions comprising other or further O-antigen conjugates, the concentration or dosage of the other or further O-antigen conjugates in typical embodiments is between 20 and 400% of the O25B antigen conjugate, preferably between 25 and 200%, e.g. between 50 and 100% of the O25B antigen conjugate. The optimal dose for administration (and corresponding concentration in a composition) for each individual O-antigen conjugate may be determined by the skilled person based upon immunological assays and in clinical trials, following the rationale and protocols described for O25B, O1A, O2 and O6A in WO 2017/035181, incorporated by reference. A typical dosage for each individual additional O-antigen conjugate in the composition for a single administration to a subject would be between 2 and 25 ug of the additional O-antigen polysaccharide in that conjugate, for example between 4 and 16 ug per polysaccharide.

Carrier Proteins

A carrier protein that can be used to conjugate the O-antigen to can be selected from any carrier proteins known to those of skill in the art, e.g., detoxified Exotoxin A of P. aeruginosa (EPA; see, e.g., Ihssen, et al., supra), FimH, flagellin (FliC), CRM197, maltose binding protein (MBP), Diphtheria toxoid, Tetanus toxoid, detoxified hemolysin A of S. aureus, clumping factor A, clumping factor B, E. coli heat labile enterotoxin, detoxified variants of E. coli heat labile enterotoxin, Cholera toxin B subunit (CTB), cholera toxin, detoxified variants of cholera toxin, E. coli Sat protein, the passenger domain of E. coli Sat protein, Streptococcus pneumoniae Pneumolysin and detoxified variants thereof, etc. Preferred examples are CRM197 and EPA, EPA being particularly preferred. In a particularly preferred embodiment, the carrier protein is EPA having the amino acid sequence of SEQ ID NO: 1.

According to certain embodiments of the invention, each E. coli O-antigen is covalently bound to an EPA carrier protein (see, e.g., Ihssen et al., supra). For EPA, various detoxified protein variants have been described in literature and could be used as carrier proteins.

In certain embodiments, the EPA carrier proteins used in the conjugates of the invention are modified in such a way that the protein is less toxic and/or more susceptible to glycosylation. For example, detoxification can be achieved by mutating and deleting the catalytically essential residues L552V and 4E553 according to Lukac et al., 1988, Infect Immun, 56: 3095-3098, and Ho et al., 2006, Hum Vaccin, 2:89-98. In a specific embodiment, the carrier proteins used in the generation of the conjugates of the invention are modified such that the number of glycosylation sites in the carrier proteins is optimized in a manner that allows for lower concentrations of the protein to be administered, e.g., in an immunogenic composition, in its bioconjugate form.

In certain embodiments, the EPA or other carrier proteins are modified to include 1, 2, 3, 4, 5, 6, 7, 8, 9, 10, or more glycosylation sites than would normally be associated with the carrier protein (e.g., relative to the number of glycosylation sites associated with the carrier protein in its native/natural, e.g., “wild-type” state). In specific embodiments, introduction of glycosylation sites is accomplished by insertion of glycosylation consensus sequences (e.g., Asn-X-Ser(Thr) (SEQ ID NO: 3), wherein X can be any amino acid except Pro; or preferably Asp(Glu)-X-Asn-Z-Ser(Thr) (SEQ ID NO: 2), wherein X and Z are independently selected from any natural amino acid except Pro (see, e.g., WO 2006/119987), anywhere in the primary structure of the protein. In one particular embodiment, the EPA carrier protein comprises 4 consensus glycosylation sequences of the sequence Asp/Glu-X-Asn-Z-Ser/Thr, and has the amino acid sequence of SEQ ID NO: 1.

An EPA carrier protein useful in the invention can be produced by methods known in the art in view of the present disclosure. See, e.g., relevant disclosure in e.g., Ihssen et al., supra, and in WO 2006/119987, WO 2009/104074, and WO 2015/124769, the disclosures of which are incorporated by reference herein. In certain embodiments, the EPA carrier protein can be produced together with a signal sequence (such as a signal peptide for E. coli DsbA, E. coli outer membrane porin A (OmpA), E. coli maltose binding protein (MalE), etc.) that targets the carrier protein to the periplasmic space of the host cell that expresses the carrier protein. The EPA carrier protein can also be modified to contain a “tag,” i.e., a sequence of amino acids that allows for the isolation and/or identification of the carrier protein.

Other carrier proteins can be made by similar means. For chemical conjugation, carrier proteins do not need the glycosylation consensus sequences mentioned above, and can typically be obtained by recombinant protein production according to methods known in the art.

According to certain embodiments of the invention, the E. coli O-antigens are covalently bound to the carrier protein via bioconjugation. Accordingly, in certain embodiments, a host cell can produce an E. coli O-antigen and an EPA carrier protein, and covalently bind the O-antigen to the EPA carrier protein to form a bioconjugate useful in the invention. See, e.g., relevant disclosure in e.g., Ihssen et al., supra, and in WO 2006/119987, WO 2009/104074, and WO 2015/124769, the disclosures of which are incorporated by reference herein.

According to an embodiment of the invention, the E. coli O-antigens are covalently bound to the carrier protein via bioconjugation at the Asn residue of a glycosylation sequence comprising Asp (Glu)-X-Asn-Z-Ser (Thr) (SEQ ID NO: 2), wherein X and Z are independently selected from any natural amino acid except Pro.

In a specific embodiment, the EPA carrier protein is N-linked to an E. coli O-antigen useful in the invention. For example, the E. coli O-antigen is linked to the Asn residue in a glycosylation sequence of a carrier protein, such as Asn-X-Ser(Thr) (SEQ ID NO: 3), wherein X can be any amino acid except Pro, preferably Asp(Glu)-X-Asn-Z-Ser(Thr) (SEQ ID NO: 2), wherein X and Z are independently selected from any natural amino acid except Pro.

According to other embodiments, the O-polysaccharides can be prepared by chemical synthesis, i.e., prepared in vitro outside of host cells. In other embodiments the (lipo)polysaccharides are purified from host cells. For example, the E. coli O-antigens of the invention and purified from host cells or chemically synthesized, can be conjugated to carrier proteins using methods known to those of skill in the art, including by means of using activation reactive groups in the polysaccharide/oligosaccharide as well as the protein carrier. See, e.g., Pawlowski et al., 2000, Vaccine, 18:1873-1885; and Robbins et al., 2009, Proc Natl Acad Sci USA, 106:7974-7978, the disclosures of which are herein incorporated by reference. Such approaches comprise chemical synthesis or extraction of antigenic polysaccharides/oligosaccharides from host cells, purifying the polysaccharides/oligosaccharides, chemically activating the polysaccharides/oligosaccharides, and conjugating the polysaccharides/oligosaccharides to a carrier protein. Preparation of O-antigens for chemical conjugation and preparation of chemical conjugates have been described in the art, e.g. U.S. Pat. No. 5,370,872; Cryz S J et al, 1995, supra; Fattom A et al, 1999, Vaccine 17: 126-133; Micoli F et al, 2013, Anal Biochem 434: 136-145; Stefanetti G et al, 2014, Vaccine 32: 6122-6129; Stefanetti G et al, 2015, Angew. Chem. Int. Ed. 54, http://dx.doi.org/10.1002/anie.201506112; Stefanetti G et. al., 2015, Bioconjug Chem 26: 2507-2513; Meloni E et al, 2015, J Biotechnol 198: 46-52; Rondini S et al, 2015, Infect Immun 83: 996-1007; Baliban S M et al, 2017, PLoS Neglected Tropical Diseases, doi.org/10.1371/journal.pntd.0005493.

Bioconjugates have advantageous properties over glycoconjugates synthesized chemically in vitro using purified polysaccharides from host cells, e.g., bioconjugates require less chemicals in manufacturing and are more consistent and homogenous in terms of the final product generated. Bioconjugates can be produced by a relatively generic process, whereas synthetic conjugates will need structure-dependent tailor-made process for each separate structure, which is an important issue especially for high valency products. Thus, bioconjugates are preferred over such chemically produced glycoconjugates.

In certain embodiments, the E. coli O-antigens are covalently bound to the carrier protein at a polysaccharide-to-protein weight/weight ratio of 1:20 to 20:1, preferably 1:10 to 10:1, more preferably of 1:3 to 3:1. In certain non-limiting embodiments for bioconjugates of O25B, O1A, O2 and O6A, the ratio of polysaccharide/protein is between about 0.1 and 0.5 (i.e. polysaccharide:protein is 1:10 to 1:2), depending on the O-antigen serotypes.

The conjugates of the invention can be purified by any method known in the art for purification of a protein, for example, by chromatography (e.g., ion exchange, anionic exchange, affinity, and sizing column chromatography), centrifugation, differential solubility, or by any other standard technique for the purification of proteins. See, e.g., Saraswat et al., 2013, Biomed. Res. Int., ID#312709 (p. 1-18); see also the methods described in WO 2009/104074. The actual conditions used to purify a particular conjugate will depend, in part, on the synthesis strategy (e.g., synthetic production vs. recombinant production) and on factors such as net charge, hydrophobicity, and/or hydrophilicity of the conjugate, and will be apparent to those having skill in the art.

FimH Polypeptide

As used herein, the terms “FimH polypeptide,” “FimH protein,” “FimH antigen,” and “FimH” all refer to a FimH adhesin polypeptide, a variant thereof, or an antigenic fragment thereof. In preferred embodiments of the invention, FimH is E. coli FimH. FimH is an adhesin that in nature can be found at the tip of type 1 fimbriae or pili on the surface of E. coli, where it facilitates adhesion and adherence to cells or surfaces such as bladder epithelial cells. FimH is responsible for D-mannose-sensitive adhesion. Mature FimH is displayed on the bacterial surface as a component of the type 1 fimbrial organelle. A “FimH” polypeptide according to the invention comprises at least part of the domain that facilitates the adhesion process, which in nature is localized toward the N-terminus. Vaccine compositions comprising FimH or fragments thereof can, upon administration, induce antibodies to FimH which can prevent or reduce bacterial adhesion and/or mediate bacterial killing via opsonophagocytosis. FimH can be purified from natural E. coli cells. In preferred embodiments, FimH is recombinantly expressed and produced in a suitable host cell such as E. coli. As used herein, the terms “FimHt” and “FimH_{LD}” refer to truncated forms of FimH, wherein a part of the C-terminus of the mature protein is deleted (e.g., Langermann et al., 1997, supra; Schembri et al., 2000, FEMS Microbiol Letters 188: 147-51; Rabbani et al., 2010, Anal Biochem 407: 188-195; Schwartz et al., 2013, Proc Natl Acad Sci USA 110: 15530-15537). In certain non-limiting embodiments, FimH polypeptide is FimHt and comprises the amino acid sequence of SEQ ID NO: 5. In another non-limiting embodiment, FimH polypeptide is FimH_{LD} and comprises the amino acid sequence of SEQ ID NO: 8. In a further non-limiting embodiment, FimH_{LD} comprises the amino acid sequence of SEQ ID NO: 9.

The compositions of the invention comprise FimH. FimH that can be used in the compositions according to the invention can be any FimH or variant thereof, including any conformation or form of FimH. Structural analysis of FimH polypeptides demonstrated the existence of different conformational states which display differential mannose-binding affinities (e.g. Le Trong et al, 2010, Cell 141: 645-655; Kalas et al, Sci Adv. 2017,Feb. 10; 3(2):e1601944, doi: 10.1126/sciadv.1601944. eCollection 2017 February, PMID: 28246638: Choudhury et al, 1999, Science 285: 1061-1066). In certain embodiments, FimH adopts a compact conformation where the mannose-binding domain is in a low-affinity state, as characterized by a shallow binding pocket. In another embodiment, the FimH polypeptide adopts an elongated conformation where the mannose-binding domain is in a high-affinity conformation, as characterized by a narrower binding pocket. In certain embodiments, FimH is truncated and displays a high affinity conformation. In certain embodiments, FimH is complexed to its chaperone FimC and exhibits a high affinity conformation. Certain amino acid substitutions localized to the mannose-binding domain have been demonstrated to affect the conformational state of truncated FimH in the absence of a ligand (e.g. Kisiela et al, 2013, Proc Natl Acad Sci USA 110: 19089-19094; Rabbani et al, 2018, J Biol Chem 293: 1835-1849). In certain embodiments, truncated FimH comprises one or more amino acid mutations that stabilize it in the low-affinity conformation, in particular in the absence of ligand (mannose). A non-limiting example of such amino acid mutations is an amino acid substitution at position 60, such as an arginine-to-proline substitution at position 60 (R60P). For this purpose, amino acid position numbering is in alignment with SEQ ID NO: 9, i.e. this can also be done for other FimH sequences by aligning such sequences with SEQ ID NO: 9 and substituting arginine at the corresponding position with proline.

In certain embodiments, FimH is a full length FimH. One example of a full length FimH (300 amino acids) sequence is provided in SEQ ID NO: 4. Other non-limiting examples are provided in SEQ ID NO: 6 (which is identical to SEQ ID NO: 2 of U.S. Pat. No. 6,500,434) and SEQ ID NO: 10.

In certain embodiments, FimH comprises a mature form of FimH, lacking part of the N-terminus of the full length FimH protein (e.g. mature FimH lacks the N-terminal signal sequence). In certain embodiments, mature FimH comprises the amino acid sequence of SEQ ID NO: 7 (which is identical to SEQ ID NO: 29 of U.S. Pat. No. 6,737,063). Another non-limiting example is SEQ ID NO: 11.

In certain embodiments, FimH is a truncated form of FimH, such as FimHt or FimH_{LD}, comprising the N-terminal amino acids of mature FimH but lacking part of the C-terminus. In certain embodiments, the truncated FimH contains amino acids 1-157, 1-160, 1-161, 1-181, 1-186, 1-196, 1-207, or 1-223 of the mature FimH protein, e.g. as disclosed in SEQ ID NO: 7. In a particular embodiment, the truncated FimH comprises the N-terminal 186 amino acids of the mature FimH protein. In another particular embodiment, the truncated FimH comprises the N-terminal 160 amino acids of the mature FimH protein. In certain embodiments, truncated FimH comprises amino acids 26 to 186 of SEQ ID NO: 7. In one embodiment, truncated FimH comprises the amino acid sequence of SEQ ID NO: 5. In another embodiment, truncated FimH comprises the amino acid sequence of SEQ ID NO: 8.

The skilled person is able to make suitable variants by deletion, addition, and/or substitution of amino acids from any of these exemplary FimH embodiments, and such variants are also considered FimH proteins according to the invention. Natural FimH variants can also be used.

In certain embodiments, FimH is stabilized by methods known in the art. In particular embodiments, the FimH is complexed with its chaperone FimC, also known as FimCH (e.g. Choudhury D et al, 1999, Science 285: 1061-1066). In other embodiments, FimH is FimH_{LD} that is combined with the FimH pilin domain (FimH-PD), representing or actually being mature or full length FimH. In other embodiments, FimH is stabilized by addition or fusion of the donor-strand peptide of FimG (DsG) (i.e., FimG residues 1-13 or 1-14) (see, e.g., Barnhart M M et al, 2000, Proc Natl Acad Sci USA, 97: 7709-7714; Sauer M M et al., 2016, Nat Commun, 7:10738) or full length FimG (e.g. Barnhart M M et al, 2003, J Bacteriol. 185: 2723-2730).

FimCH and truncated FimH have been shown to be capable of generating antibodies and effective immune responses against E. coli UTI in preclinical models (e.g. Langermann S, et al., 1997 and 2000, supra; O'Brien V P et al., supra). Indeed, the company Sequoia Sciences reported that an investigational vaccine consisting of FimH protein and an adjuvant was highly immunogenic and well-tolerated and may reduce the frequency of UTI, based on preliminary results from a phase 1 clinical trial in women (https://www.sequoiasciences.com/uti-vaccine-program).

FimH in the compositions of the invention can be isolated from bacterial pili that naturally comprise FimH at their tips. In certain embodiments, FimH is chemically synthesized, or in other embodiments, FimH is synthesized by in vitro or ex vivo protein biosynthesis. In preferred embodiments, FimH is recombinantly expressed, e.g. in bacterial cells that have been transformed with nucleic acid encoding FimH under control of a promoter that drives expression of FimH, according to methods known in the art. For instance, DNA encoding FimH or part of FimH can be cloned into an expression vector, and after transformation of a suitable host cell, such as E. coli, the FimH can be expressed and purified from the host cells or culture medium according to standard methods known in the art. Examples of recombinant expression of FimH can for instance be found in WO 2002/004496, and Rabbani S et al, 2010, Anal Biochem 407: 188-195, each incorporated by reference herein. In certain embodiments, FimH may be linked to a polypeptide tag, e.g. for purification or recognition, e.g. a His-tag.

In certain embodiments, the FimH is administered to humans at about 1 to about 200 ug per administration (dose), e.g. 1-150 ug per dose, e.g. about 1, 5, 10, 15, 20, 25, 30, 40, 50, 60, 70, 80, 90, 100, 110, 120, 130, 140 or 150 ug per dose. In certain embodiments, compositions of the invention comprise FimH at a concentration of about 2-400 ug/mL (e.g. for administration of single doses of 0.5 mL), e.g. about 2-200 ug/mL, e.g. about 2, 5, 10, 20, 30, 40, 50, 60, 70, 80, 90, 100, 110, 120, 130, 140, 150, 160, 170, 180, 190, or 200 ug/mL. For dosages of FimH also see, e.g., US20030138449.

Adjuvant

As used herein, the term “adjuvant” refers to a compound that when administered in conjunction with or as part of a composition of the invention augments, enhances and/or boosts the immune response to a conjugate comprising E. coli O-antigen coupled to a carrier protein and/or to FimH, but when the adjuvant compound is administered alone does not generate an immune response to the conjugate and/or FimH. Adjuvants can enhance an immune response by several mechanisms including, e.g., lymphocyte recruitment, stimulation of B and/or T cells, and stimulation of antigen presenting cells.

The compositions of the invention (e.g., the immunogenic compositions) comprise, or are administered in combination with, an adjuvant. The adjuvant for administration in combination with a composition of the invention can be administered before, concomitantly with, or after administration of the immunogenic compositions.

Specific examples of adjuvants include, but are not limited to, aluminum salts (alum) (such as aluminum hydroxide, aluminum phosphate, aluminum sulfate, and aluminum oxide, including nanoparticles comprising alum or nanoalum formulations), calcium phosphate (e.g. Masson J D et al, 2017, Expert Rev Vaccines 16: 289-299), monophosphoryl lipid A (MPL) or 3-de-O-acylated monophosphoryl lipid A (3D-MPL) (see e.g., United Kingdom Patent GB2220211, EP0971739, EP1194166, U.S. Pat. No. 6,491,919), AS01, AS02, AS03 and AS04 (all GlaxoSmithKline; see e.g. EP1126876, U.S. Pat. No. 7,357,936 for AS04, EP0671948, EP0761231, U.S. Pat. No. 5,750,110 for AS02), imidazopyridine compounds (see WO2007/109812), imidazoquinoxaline compounds (see WO2007/109813), delta-inulin (e.g. Petrovsky N and PD Cooper, 2015, Vaccine 33: 5920-5926), STING-activating synthetic cyclic-di-nucleotides (e.g. US20150056224), combinations of lecithin and carbomer homopolymers (e.g. U.S. Pat. No. 6,676,958), and saponins, such as Quil A and QS21 (see e.g. Zhu D and W Tuo, 2016, Nat Prod Chem Res 3: e113 (doi:10.4172/2329-6836.1000e113), optionally in combination with QS7 (see Kensil et al., in Vaccine Design: The Subunit and Adjuvant Approach (eds. Powell & Newman, Plenum Press, N Y, 1995); U.S. Pat. No. 5,057,540). In some embodiments, the adjuvant is Freund's adjuvant (complete or incomplete). In certain embodiments, the adjuvant comprises Quil-A, such as for instance commercially obtainable from Brenntag (now Croda) or Invivogen. QuilA contains the water-extractable fraction of saponins from the Quillaja saponaria Molina tree. These saponins belong to the group of triterpenoid saponins, that have a common triterpenoid backbone structure. Saponins are known to induce a strong adjuvant response to T-dependent as well as T-independent antigens, as well as strong cytotoxic CD8+ lymphocyte responses and potentiating the response to mucosal antigens. They can also be combined with cholesterol and phospholipids, to form immunostimulatory complexes (ISCOMs), wherein QuilA adjuvant can activate both antibody-mediated and cell-mediated immune responses to a broad range of antigens from different origens. Certain adjuvants comprise emulsions, which are mixtures of two immiscible fluids, e.g. oil and water, one of which is suspended as small drops inside the other, and are stabilized by surface-active agents. Oil-in-water emulsions have water forming the continuous phase, surrounding small droplets of oil, while water-in-oil emulsions have oil forming the continuous phase. Certain emulsions comprise squalene (a metabolizable oil). Certain adjuvants comprise block copolymers, which are copolymers formed when two monomers cluster together and form blocks of repeating units. An example of a water in oil emulsion comprising a block copolymer, squalene and a microparticulate stabilizer is TiterMax®, which can be commercially obtained from Sigma-Aldrich. Optionally emulsions can be combined with or comprise further immunostimulating components, such as a TLR4 agonist. Certain adjuvants are oil in water emulsions (such as squalene or peanut oil) also used in MF59 (see e.g. EP0399843, U.S. Pat. Nos. 6,299,884, 6,451,325) and AS03, optionally in combination with immune stimulants, such as monophosphoryl lipid A and/or QS21 such as in AS02 (see Stoute et al., 1997, N. Engl. J. Med. 336, 86-91). Further examples of adjuvants are liposomes containing immune stimulants such as MPL and QS21 such as in ASO lE and AS01B (e.g. US 2011/0206758). Other examples of adjuvants are CpG (Bioworld Today, Nov. 15, 1998) and imidazoquinolines (such as imiquimod and R848). See, e.g., Reed G, et al., 2013, Nature Med, 19: 1597-1608.

In certain preferred embodiments, the adjuvant comprises saponins, preferably the water-extractable fraction of saponins obtained from Quillaja saponaria. In certain embodiments, the adjuvant comprises QS-21.

In certain preferred embodiments, the adjuvant contains a toll-like receptor 4 (TLR4) agonist. TLR4 agonists are well known in the art, see e.g. Ireton G C and SG Reed, 2013, Expert Rev Vaccines 12: 793-807. In certain preferred embodiments, the adjuvant is a TLR4 agonist comprising lipid A, or an analog or derivative thereof.

The adjuvant, preferably including a TLR4 agonist, may be formulated in various ways, e.g. in emulsions such as water-in-oil (w/o) emulsions or oil-in-water (o/w) emulsions (examples are MF59, AS03), stable (nano-)emulsions (SE), lipid suspensions, liposomes, (polymeric) nanoparticles, virosomes, alum adsorbed, aqueous formulations (AF), and the like, representing various delivery systems for immunomodulatory molecules in the adjuvant and/or for the immunogens (see e.g. Reed et al, 2013, supra; Alving C R et al, 2012, Curr Opin Immunol 24: 310-315).

The immunostimulatory TLR4 agonist may optionally be combined with other immunomodulatory components, such as saponins (e.g. QuilA, QS7, QS21, Matrix M, Iscoms, Iscomatrix, etc), aluminum salts, activators for other TLRs (e.g. imidazoquinolines, flagellin, CpG, dsRNA analogs, etc), and the like (see e.g. Reed et al, 2013, supra).

As used herein, the term “lipid A” refers to the hydrophobic lipid moiety of an LPS molecule that comprises glucosamine and is linked to keto-deoxyoctulosonate in the inner core of the LPS molecule through a ketosidic bond, which anchors the LPS molecule in the outer leaflet of the outer membrane of Gram-negative bacteria. For an overview of the synthesis of LPS and lipid A structures, see, e.g., Raetz, 1993, J. Bacteriology 175:5745-5753, Raetz C R and C Whitfield, 2002, Annu Rev Biochem 71: 635-700; U.S. Pat. Nos. 5,593,969 and 5,191,072. Lipid A, as used herein includes naturally occurring lipid A, mixtures, analogs, derivatives and precursors thereof. The term includes monosaccharides, e.g., the precursor of lipid A referred to as lipid X; disaccharide lipid A; hepta-acyl lipid A; hexa-acyl lipid A; penta-acyl lipid A; tetra-acyl lipid A, e.g., tetra-acyl precursor of lipid A, referred to as lipid WA; dephosphorylated lipid A; monophosphoryl lipid A; diphosphoryl lipid A, such as lipid A from Escherichia coli and Rhodobacter sphaeroides. Several immune activating lipid A structures contain 6 acyl chains. Four primary acyl chains attached directly to the glucosamine sugars are 3-hydroxy acyl chains usually between 10 and 16 carbons in length. Two additional acyl chains are often attached to the 3-hydroxy groups of the primary acyl chains. E. coli lipid A, as an example, typically has four C14 3-hydroxy acyl chains attached to the sugars and one C12 and one C14 attached to the 3-hydroxy groups of the primary acyl chains at the 2′ and 3′ position, respectively.

As used herein, the term “lipid A analog or derivative” refers to a molecule that resembles the structure and immunological activity of lipid A, but that does not necessarily naturally occur in nature. Lipid A analogs or derivatives may be modified to e.g. be shortened or condensed, and/or to have their glucosamine residues substituted with another amine sugar residue, e.g. galactosamine residues, to contain a 2-deoxy-2-aminogluconate in place of the glucosamine-1-phosphate at the reducing end, to bear a galacturonic acid moiety instead of a phosphate at position 4′. Lipid A analogs or derivatives may be prepared from lipid A isolated from a bacterium, e.g., by chemical derivation, or chemically synthesized, e.g. by first determining the structure of the preferred lipid A and synthesizing analogs or derivatives thereof. Lipid A analogs or derivatives are also useful as TLR4 agonist adjuvants (see, e.g. Gregg K A et al, 2017, MBio 8, eDD492-17, doi: 10.1128/mBio.00492-17).

For example, a lipid A analog or derivative can be obtained by deacylation of a wild-type lipid A molecule, e.g., by alkali treatment. Lipid A analogs or derivatives can for instance be prepared from lipid A isolated from bacteria. Such molecules could also be chemically synthesized. Another example of lipid A analogs or derivatives are lipid A molecules isolated from bacterial cells harboring mutations in, or deletions or insertions of enzymes involved in lipid A biosynthesis and/or lipid A modification.

MPL and 3D-MPL are lipid A analogs or derivatives that have been modified to attenuate lipid A toxicity. Lipid A, MPL and 3D-MPL have a sugar backbone onto which long fatty acid chains are attached, wherein the backbone contains two 6-carbon sugars in glycosidic linkage, and a phosphoryl moiety at the 4 position. Typically, five to eight long chain fatty acids (usually 12-14 carbon atoms) are attached to the sugar backbone. Due to derivation of natural sources, MPL or 3D-MPL may be present as a composite or mixture of a number of fatty acid substitution patterns, e.g. hepta-acyl, hexa-acyl, penta-acyl, etc., with varying fatty acid lengths. This is also true for some of the other lipid A analogs or derivatives described herein, however synthetic lipid A variants may also be defined and homogeneous. MPL and its manufacture are for instance described in U.S. Pat. No. 4,436,727. 3D-MPL is for instance described in U.S. Pat. No. 4,912,094B1, and differs from MPL by selective removal of the 3-hydroxymyristic acyl residue that is ester linked to the reducing-end glucosamine at position 3 (compare for instance the structure of MPL in column 1 vs 3D-MPL in column 6 of U.S. Pat. No. 4,912,094B1). In the art often 3D-MPL is used, while sometimes referred to as MPL (e.g. the first structure in Table 1 of Ireton G C and SG Reed, 2013, supra, refers to this structure as MPL®, but actually depicts the structure of 3D-MPL).

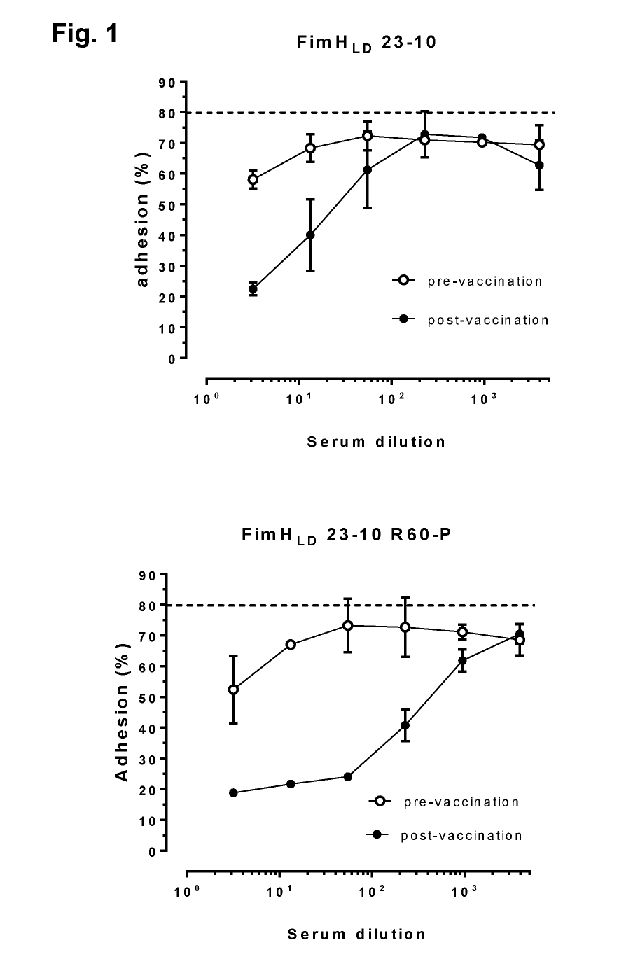

Examples of lipid A (analogs, derivatives) according to the invention include MPL, 3D-MPL, RC529 (e.g. EP1385541), PET-lipid A, GLA (glycopyranosyl lipid adjuvant, a synthetic disaccharide glycolipid; e.g. US20100310602, U.S. Pat. No. 8,722,064), SLA (e.g. Carter D et al, 2016, Clin Transl Immunology 5: e108 (doi: 10.1038/cti.2016.63), which describes a structure-function approach to optimize TLR4 ligands for human vaccines), PHAD (phosphorylated hexaacyl disaccharide; the structure of which is the same as that of GLA), 3D-PHAD, 3D-(6-acyl)-PHAD (3D(6A)-PHAD) (PHAD, 3D-PHAD, and 3D(6A)PHAD are synthetic lipid A variants, see e.g. avantilipids.com/divisions/adjuvants, which also provide structures of these molecules), E6020 (CAS Number 287180-63-6), ONO4007, OM-174, and the like. For exemplary chemical structures of 3D-MPL, RC529, PET-lipid A, GLA/PHAD, E6020, ONO4007, and OM-174, see e.g. Table 1 in Ireton G C and SG Reed, 2013, supra. For a structure of SLA, see e.g. FIG. 1 in Reed S G et al, 2016, Curr Opin Immunol 41: 85-90. In certain preferred embodiments, the TLR4 agonist adjuvant comprises a lipid A analog or derivative chosen from 3D-MPL, GLA, or SLA.

Exemplary adjuvants comprising a lipid A analog or derivative include GLA-LSQ (synthetic MPL [GLA], QS21, lipids formulated as liposomes), SLA-LSQ (synthetic MPL [SLA], QS21, lipids, formulated as liposomes), GLA-SE (synthetic MPL [GLA], squalene oil/water emulsion), SLA-SE (synthetic MPL [SLA], squalene oil/water emulsion), SLA-Nanoalum (synthetic MPL [SLA], aluminum salt), GLA-Nanoalum (synthetic MPL [GLA], aluminum salt), SLA-AF (synthetic MPL [SLA], aqueous suspension), GLA-AF (synthetic MPL [GLA], aqueous suspension), SLA-alum (synthetic MPL [SLA], aluminum salt), GLA-alum (synthetic MPL [GLA], aluminum salt), and several of the GSK ASxx series of adjuvants, including AS01 (MPL, QS21, liposomes), AS02 (MPL, QS21, oil/water emulsion), AS25 (MPL, oil/water emulsion), AS04 (MPL, aluminum salt), and AS15 (MPL, QS21, CpG, liposomes). See, e.g., WO 2013/119856, WO 2006/116423, U.S. Pat. Nos. 4,987,237, 4,436,727, 4,877,611, 4,866,034, 4,912,094, 4,987,237, 5,191,072, 5,593,969, 6,759,241, 9,017,698, 9,149,521, 9,149,522, 9,415,097, 9,415,101, 9,504,743, Reed G, et al., 2013, supra, Johnson et al., 1999, J Med Chem, 42:4640-4649, and Ulrich and Myers, 1995, Vaccine Design: The Subunit and Adjuvant Approach; Powell and Newman, Eds.; Plenum: New York, 495-524.

Non-glycolipid molecules may also be used as TLR4 agonist adjuvants, e.g. synthetic molecules such as Neoseptin-3 or natural molecules such as LeIF, see e.g. Reed S G et al, 2016, supra.

Excipients and Carriers

The compositions of the invention are useful in the treatment and prevention of UTI in subjects (e.g., human subjects) by E. coli. In certain embodiments, in addition to comprising one or more E. coli O-antigens covalently bound to a carrier protein, FimH and adjuvant, the compositions of the invention comprise a pharmaceutically acceptable carrier. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's pharmaceutical sciences,” XIII ed. Editor-in-Chief Eric W. Martin. Mack Publishing Co., Easton, Pa., 1965.

In certain embodiments, the compositions of the invention additionally comprise one or more buffers, e.g., Tris-buffered saline, phosphate buffer, and sucrose phosphate glutamate buffer.

In certain embodiments, the compositions of the invention additionally comprise one or more salts, e.g., Tris-hydrochloride, sodium chloride, calcium chloride, potassium chloride, sodium phosphate, monosodium glutamate, and aluminum salts (e.g., aluminum hydroxide, aluminum phosphate, potassium aluminum sulfate, or a mixture of such aluminum salts). In one embodiment, a composition of the invention comprises the bioconjugates of the invention in a Tris-buffered saline (TBS) pH 7.4 (e.g. containing Tris, NaCl and KCl, e.g. at 25 mM, 137 mM and 2.7 mM, respectively). In other embodiments, the compositions of the invention comprise bioconjugates of the invention in about 10 mM KH₂PO₄/Na₂HPO₄ buffer at pH of about 7.0, about 5% (w/v) sorbitol, about 10 mM methionine, and about 0.02% (w/v) polysorbate 80. In other embodiments, the compositions of the invention comprise bioconjugates of the invention in about 10 mM KH₂PO₄/Na₂HPO₄ buffer at pH of about 7.0, about 8% (w/v) sucrose, about 1 mM EDTA, and about 0.02% (w/v) polysorbate 80.

The compositions of the invention can be used for eliciting an immune response in a host to whom the composition is administered, i.e., are immunogenic. Thus, the compositions of the invention can be used as vaccines against UTI, and can comprise any additional components suitable for use in a vaccine.

In certain embodiments, the compositions of the invention additionally comprise a preservative, such as phenol, benzethonium chloride, 2-phenoxyethanol, or thimerosal. In a specific embodiment, the pharmaceutical compositions of the invention comprise 0.001% to 0.01% preservative. In other embodiments, the pharmaceutical compositions of the invention do not comprise a preservative.

Vaccine Combinations/Compositions

In a specific embodiment, the vaccine combinations of the invention contain multivalent formulations, e.g., at least tetravalent (with respect to O-antigen serotype) formulations comprising bioconjugates of E. coli O-antigens of the O25B, O1A, O6A, and O2 serotypes/subserotypes, FimH, and adjuvant, in the same or different compositions.

The invention relates to a vaccine combination, preferably a multivalent vaccine, comprising (i) a FimH polypeptide, (ii) one or more conjugates comprising an E. coli O-antigen polysaccharide covalently coupled to a carrier protein, and (iii) an adjuvant. In one embodiment, the vaccine combination comprises a first composition comprising (i), a second composition comprising (ii), and a third composition comprising (iii), i.e. each of components (i)-(iii) of the combination is present in a separate composition. In another embodiment, the vaccine combination comprises a first composition comprising (i) and (ii) and a second composition comprising (iii). In another embodiment, the vaccine combination comprises a first composition comprising (i) and (iii) and a second composition comprising (ii). In another embodiment, the vaccine combination comprises a first composition comprising (i) and a second composition comprising (ii) and (iii). In a preferred embodiment, the vaccine combination comprises a composition comprising (i), (ii) and (iii). Such an embodiment preferably comprises a stable composition comprising (i), (ii) and (iii), but alternatively if such a composition would not be stable for longer periods, such composition can be created by mixing the components just before administration to a subject in a mix-and-shoot immunization procedure. The compositions may further comprise a pharmaceutically acceptable carrier. If the components (i), (ii) and (iii) are not present in a single composition they may be administered in combination to a subject. When there is more than one conjugate in the combination, these conjugates are preferably present in a single composition.

In a specific embodiment, a vaccine combination provided herein contains a composition comprising: (a) FimH, (b,i) an E. coli O25B bioconjugate comprising an E. coli O25B antigen covalently bound to an EPA carrier protein; (b,ii) an E. coli O1A bioconjugate comprising an E. coli O1A antigen covalently bound to an EPA carrier protein; (b,iii) an E. coli O2 bioconjugate comprising an E. coli O2 antigen covalently bound to an EPA carrier protein; and (b,iv) an E. coli O6A bioconjugate comprising an E. coli O6A antigen covalently bound to an EPA carrier protein, and (c) an adjuvant. Again such composition may be preformulated during manufacturing wherein all components are present in a single composition, or alternatively such composition may be prepared by mixing compositions comprising one or more of the components just prior to use.

In certain embodiments, the compositions of the invention are formulated to be suitable for the intended route of administration to a subject. For example, the compositions of the invention can be formulated to be suitable for subcutaneous, parenteral, oral, intradermal, transdermal, colorectal, intraperitoneal, intravaginal, or rectal administration. In a specific embodiment, the pharmaceutical composition can be formulated for intravenous, oral, buccal, intraperitoneal, intranasal, intratracheal, subcutaneous, intramuscular, topical, intradermal, transdermal or pulmonary administration, preferably intramuscular administration.

The compositions of the invention can be included in a container, pack, or dispenser together with instructions for administration.

In certain embodiments, the compositions of the invention can be stored before use, e.g., the compositions can be stored frozen (e.g., at about −20° C. or at about −70° C.); stored in refrigerated conditions (e.g., at about 2-8° C., e.g. about 4° C.); or stored at room temperature. Alternatively, separate compositions comprising one or more of the components (i), (ii), and (iii) may be stored and mixed to the vaccine combination composition comprising all three of (i), (ii) and (iii) prior to use. In yet another alternative, the separate compositions are provided in a combination administration schedule.

Methods and Uses

In another general aspect, the invention relates to a method of inducing an immune response to E. coli in a subject in need thereof. Preferably, the immune response is effective to prevent or treat one or more symptoms associated with UTI in the subject in need thereof. The method comprises administering to the subject one or more conjugates comprising one or more E. coli O-antigens covalently coupled to one or more carrier proteins, FimH polypeptide, and adjuvant. The conjugate, FimH and adjuvant and aspects thereof are as described above.

Preferably, the at least one E. coli O-antigen used in the methods and uses of the invention is prevalent among the E. coli clinical isolates causing UTI, as described above, such as an E. coli O25B antigen.

The conjugates comprising the O-antigens are capable of inducing the production of opsonophagocytic antibodies against E. coli in a subject in need thereof, see e.g. WO 2015/124769 and WO 2017/035181.

In a specific embodiment, the methods of inducing an immune response in a subject of the invention result in vaccination of the subject to induce a protective immunity against infection by the E. coli strains whose O-antigens are present in the composition(s). When an O-antigen subtype is used, a method of the invention can also induce immune response to another O-antigen subtype having similar antigenicity.

In a specific embodiment, the immune response induced by a method or composition of the invention is effective to prevent and/or reduce the incidence of at least a UTI caused by E. coli of the O25 serotype (e.g. O25B and/or O25A), and the following E. coli serotypes: O1 (e.g., O1A, O1B, and/or O1C), O2, and/or O6 (e.g., O6A and/or O6GlcNAc).

In order to immunize a subject against a UTI, or treat a subject having a UTI, the subject can be administered a single composition of the invention, wherein the composition comprises at least one E. coli O-antigen, and optionally one, two, three, four, five, six, seven, eight, nine, ten, eleven or more additional E. coli O-antigens, each covalently bound to a carrier protein such as EPA, and FimH polypeptide and adjuvant. Alternatively, in order to treat a subject having a UTI, or immunize a subject against a UTI, the subject can be administered multiple compositions of the invention in combination together comprising one or more conjugates comprising one or more E. coli O-antigens covalently coupled to a carrier protein, FimH polypeptide and adjuvant. For example, a subject can be administered a composition comprising FimH and E. coli O-antigens conjugated to carrier proteins, in combination with the administration of a composition comprising an adjuvant. In embodiments where a subject is administered multiple compositions of the invention in combination, it is preferred to administer the multiple compositions within a time frame and at a location that allows draining of the vaccine combination components to the same lymph node, e.g. by administering the compositions in the same limb within short distance, e.g. within 30 cm, 20 cm, within 10 cm, within 5 cm, within 2 cm of each other, and within a few days of each other, e.g. within 72 hours, 48 hours, 24 hours, 8 hours, 2 hours, 1 hour. Most practical is administration, e.g. by intramuscular injection, in one session, e.g. within 30 minutes, within 10 minutes, preferably within 5 minutes, within 2 minutes, preferably co-administration essentially simultaneously.

In certain embodiments, the immune response induced in a subject following administration of a composition of the invention is effective to eliminate a UTI.

In certain embodiments, the immune response induced in a subject following administration of a composition of the invention is effective to prevent or reduce a symptom of UTI, preferably in at least 30%, more preferably at least 40%, such as at least 50%, of the subjects administered with the composition. Symptoms of UTI can vary depending on the nature of the infection and can include, but are not limited to: dysuria, increased urinary frequency or urgency, pyuria, hematuria, back pain, pelvic pain, pain while urinating, fever, chills, and/or nausea.

In certain embodiments, the immune response induced in a subject following administration of a composition of the invention is effective to prevent or reduce organ failure resulting from a UTI. In certain embodiments, the immune response induced in a subject following administration of a composition of the invention is effective to reduce the likelihood of hospitalization of a subject suffering from a UTI. In some embodiments, the immune response induced in a subject following administration of a composition of the invention is effective to reduce the duration of hospitalization of a subject suffering from a UTI.

Combination Therapies

In certain embodiments, a composition of the invention is administered to a subject in combination with one or more other therapies (e.g., antibacterial or immunomodulatory therapies). The one or more other therapies can be beneficial in the treatment or prevention of UTI or can ameliorate a symptom or condition associated with a UTI. In some embodiments, the one or more other therapies include administration of antibiotics useful for treating UTIs. In some embodiments, the one or more other therapies are pain relievers or anti-fever medications. In certain embodiments, the therapies are administered less than 5 minutes apart to less than 1 week apart. Any anti-bacterial agents known to one of skill in the art (e.g. antibiotics) can be used in combination with a composition of the invention.

In certain embodiments, the immune response induced in a subject following administration of a composition of the invention is effective to enhance or improve the prophylactic or therapeutic effect(s) of another therapy.

Dosage and Frequency of Administration

Administration of the compositions of the invention can be done via various routes known to the clinician, for instance subcutaneous, parenteral, intravenous, intramuscular, topical, oral, intradermal, transdermal, intranasal, etc. In one embodiment, administration is via intramuscular injection.

As used herein in the context of administering an O-antigen or FimH to a subject using methods according to embodiments of the invention, the term “effective amount” refers to the amount of the O-antigen or FimH that is sufficient to induce a desired immune effect or immune response in the subject. In certain embodiments, an “effective amount” refers to the amount of an O-antigen and FimH which is sufficient to produce immunity in a subject to achieve one or more of the following effects in the subject: (i) prevent the development or onset of a UTI or symptom associated therewith; (ii) prevent or reduce the recurrence of a UTI or symptom associated therewith; (iii) prevent, reduce or ameliorate the severity of a UTI or symptom associated therewith; (iv) reduce the duration of infection UTI or symptom associated therewith; (v) prevent the clinical progression of a UTI or symptom associated therewith; (vi) cause regression of a UTI or symptom associated therewith; (vii) prevent or reduce organ failure resulting from UTI; (viii) reduce the chance or frequency of hospitalization of a subject having a UTI; (ix) reduce hospitalization length of a subject having a UTI; (x) eliminate a UTI; and/or (xi) enhance or improve the prophylactic or therapeutic effect(s) of another therapy.

Selection of a particular effective dose can be determined (e.g., via clinical trials) by those skilled in the art based upon the consideration of several factors, including the disease to be treated or prevented, the symptoms involved, the medical history of the subject, the physical condition of the subject, such as the subject's age, weight and/or immune status, the composition administered, such as the target O-antigens, FimH polypeptide, adjuvant, etc., and other factors known by the skilled artisan. The precise dose to be employed in the formulation will also depend on the route of administration, such as oral or parenteral, and the severity of disease, and should be decided according to the judgment of the practitioner and each patient's circumstances. Effective doses can be extrapolated from dose-response curves derived from in vitro or animal model test systems. Guidance for possible dose ranges for the O-antigen conjugates and for the FimH components of the vaccine compositions is provided hereinabove.

In certain embodiments of the invention, a subject in need thereof is administered with 0.5 mL of a composition according to the invention.

In certain embodiments, an exemplary dosage for per administration to a human subject corresponds to 0.5 mL of a composition containing a first concentration of about 1-50 ug/mL, e.g. about 8-48 ug/mL, e.g., about 8, 12, 16, 20, 24, 28, 32, 36, 40, 44 or 48 ug/mL, of E. coli O25B antigen covalently bound to an EPA carrier protein, a concentration of 20% to 200% of the first concentration for each of one or more additional E. coli O-antigens covalently bound to the EPA carrier protein, and a concentration of about 1-200 ug/mL, e.g. about 1-100 ug/mL, e.g., about 1, 2, 4, 8, 12, 16, 20, 30, 40, 50, 60, 70, 80, 90 or 100 ug/mL of FimH. In certain embodiments, the adjuvant contains a TLR4 agonist, e.g. MPL, 3D-MPL, RC529, GLA, SLA, E6020, PET-lipid A, PHAD, 3D-PHAD, 3D-(6-acyl)-PHAD, ONO4007, OM-174, or the like, any of these optionally formulated in oil-in-water (A502-like) or in liposomes (AS01-like), with or without the saponin QS21. Optimal dosages for the TLR4 agonist adjuvant components can be determined by the skilled person according to well-known methods that are routine for the practitioner, and can in exemplary embodiments for instance be between 0.1 and 1000, typically between 1 and 100, e.g. 1, 2, 3, 4, 5, 6, 7, 8, 9, 10, 15, 20, 25, 30, 40, 50, 60, 70, 80, 90 or 100 ug of TLR4 agonist component per administration.

In certain embodiments, a composition or a vaccine combination of its constituents as separate compositions of the invention is administered to a subject once as a single dose. In certain embodiments, a composition of the invention or a vaccine combination of its constituents as separate compositions is administered to a subject as a single dose followed by a second dose 3 to 8 weeks later. In accordance with certain embodiments, booster inoculations can optionally be administered to the subject at 6 to 24 month intervals following the first or second inoculation. In certain embodiments, the booster inoculations can utilize a different E. coli O-antigen, bioconjugate, FimH polypeptide, adjuvant, or composition. In certain embodiments, a composition of the invention is administered to a subject as a single dose once per n years, n being for instance about 1, 2, 3, 4, 5, 6, 7, 8, 9, 10, 15 or 20.

Patient Populations

In certain embodiments, a composition or method of the invention is administered or applied to a naïve subject, i.e., a subject that does not have an E. coli infection or has not previously had a UTI. In one embodiment, a composition or method of the invention is administered or applied to a subject that is at risk of acquiring or developing a UTI, e.g., an immunocompromised or immunodeficient individual, before symptoms manifest or symptoms become severe. In certain embodiments, a composition or method of the invention is administered or applied to a subject who has been or was previously diagnosed with a UTI.

As used herein, the term “at-risk human” refers to a human that is more prone to a condition than the average human adult population. Examples of an “at-risk human” include persons that have one or more risk factors for UTI which can include, but are not limited to, elderly people, immunocompromised people, people with diabetes, people with known history of rUTI, people with obstructions in the urinary tract such as kidney stones, sexually active women, women after menopause, people using a catheter, people that are incontinent, people recently having undergone a urinary system procedure such as surgery on the urinary tract, etc.

In certain embodiments, a composition or method of the invention is administered or applied to a subject who has been or was previously diagnosed with a UPEC infection. In some embodiments, a composition or method of the invention is administered or applied to a subject suffering from reoccurring UTIs. In some embodiments, a composition or method of the invention is administered or applied to a subject suffering from reoccurring UTIs, but is healthy at the moment of treatment. In some embodiments, a composition or method of the invention is administered or applied to a subject having or at risk of acquiring E. coli bacteremia or sepsis. In some embodiments, a subject to be administered or applied a composition or method of the invention has a condition that requires them to use a catheter, such as a urinary catheter (which leads to risk of CAUTI, i.e. catheter associated UTI). In some embodiments, a composition or method of the invention is administered or applied to a subject that undergoes a pre-scheduled surgery.

In some embodiments, a subject to be administered or applied a composition or method of the invention is an animal. In certain embodiments, the animal is a mammal, e.g., a horse, swine, rabbit, mouse, or primate. In a preferred embodiment, the subject is a human.

In certain embodiments, a subject to be administered or applied a composition or method of the invention is a human subject, preferably, a human subject at risk of having disease UTI. In certain embodiments, a subject to be administered or applied a composition or method of the invention is a human adult more than 50 years old. In certain embodiments, a subject to be administered or applied a composition or method of the invention is a human adult more than 55, more than 60 or more than 65 years old.

In certain embodiments, a subject to be administered or applied a composition or method of the invention is a woman between age of about 16 to 50 years old, e.g. between age of about 16 and 35 years old.

In certain embodiments, a subject to be administered or applied a composition or method of the invention has diabetes.

### Assays

The ability of the compositions of the invention to generate an immune response in a subject can be assessed using any approach known to those of skill in the art in view of the present disclosure, and for instance described in WO 2015/124769 and WO 2017/035181.

Animal models for testing efficacy of compositions of the invention to prevent UTI have been described for instance in Langermann S, et al., 1997 and 2000, supra, and O'Brien V P et al., 2016, supra, the disclosures of which are incorporated by reference herein.

### Kits

Provided herein is a pack or kit comprising one or more containers filled with one or more of the ingredients of the compositions of the invention, such as one or more E. coli O-antigens and/or conjugates of the E. coli O-antigens covalently bound to a carrier protein according to embodiments of the invention, or FimH polypeptide, or adjuvant. Optionally associated with such container(s) can be a notice or instructions in the form prescribed by a governmental agency regulating the manufacture, use or sale of pharmaceuticals or biological products, which notice reflects approval by the agency of manufacture, use or sale for human administration. The kits encompassed herein can be used in the above methods of treatment and immunization of subjects.

### Embodiments

The invention provides also the following non-limiting embodiments.

Embodiment 1 is a vaccine combination comprising a FimH polypeptide, one or more conjugates comprising an E. coli O-antigen polysaccharide covalently coupled to a carrier protein, and an adjuvant.

Embodiment 2 is the vaccine combination of Embodiment 1, wherein the one or more conjugates comprise E. coli O25B antigen polysaccharide.

Embodiment 3 is the vaccine combination of Embodiment 2, wherein the one or more conjugates further comprise E. coli O1A antigen polysaccharide, E. coli O2 antigen polysaccharide, and E. coli O6A antigen polysaccharide.

Embodiment 4 is the vaccine combination of any one of Embodiments 2 or 3, wherein the one or more conjugates further comprise 1, 2, 3, 4, 5, 6, 7, 8, 9, 10, 11, 12, 13, 14, 15, 16, 17, 18, 19, or 20 further E. coli antigen polysaccharides.

Embodiment 5 is the vaccine combination of Embodiment 4, wherein the 1-16 further E. coli antigen polysaccharides include one or more of O4, O7, O9, O11, O12, O22, O75, O8, O15, O16, or O18.

Embodiment 6 is the vaccine combination of any one of Embodiments 1-5, wherein the one or more conjugates are bioconjugates.

Embodiment 7 is the vaccine combination of any one of Embodiments 3-6, wherein the amount of each of the further E. coli polysaccharides is 20-100% of the amount of the E. coli O25 antigen polysaccharide.

Embodiment 8 is the vaccine combination of any one of Embodiments 2 to 7, comprising 1-50 ug/mL of each of the O antigen polysaccharides.

Embodiment 9 is the vaccine combination of any one of Embodiments 1 to 8, wherein the carrier protein is detoxified exotoxin A of Pseudomonas aeruginosa (EPA).

Embodiment 10 is the vaccine combination of Embodiment 9, wherein the E. coli O-antigen polysaccharide is linked to the Asn residue of Asn-X-Ser(Thr) (SEQ ID NO: 3), preferably Asp(Glu)-X-Asn-Z-Ser(Thr) (SEQ ID NO: 2), in the EPA, wherein X and Z are independently selected from any natural amino acid except Pro.

Embodiment 11 is the vaccine combination of Embodiment 9 or 10, wherein the EPA has the amino acid sequence of SEQ ID NO: 1.

Embodiment 12 is the vaccine combination of any one of Embodiments 1 to 11, wherein the FimH polypeptide comprises a truncated form of FimH.

Embodiment 13 is the vaccine combination of any one of Embodiments 1 to 11, wherein the FimH polypeptide comprises FimCH.

Embodiment 14 is the vaccine combination of any one of the Embodiments 1 to 11, wherein FimH is the mature FimH polypeptide.

Embodiment 15 is the vaccine combination of Embodiment 14, wherein the mature FimH polypeptide is stabilized by FimG or by a donor-strand peptide of FimG (DsG).

Embodiment 16 is the vaccine combination of Embodiment 15, wherein the donor-strand peptide of FimG (DsG) is fused to mature FimH via a flexible linker.

Embodiment 17 is the vaccine combination of any one of Embodiments 1-16, comprising about 2-200 ug/mL of FimH polypeptide.

Embodiment 18 is the vaccine combination of any one of Embodiments 1-12, wherein the FimH polypeptide comprises amino acids 1-157, 1-160, 1-161, 1-181, 1-186, 26-186, 1-196, 1-207, or 1-223 of SEQ ID NO: 7.

Embodiment 19 is the vaccine combination of any one of Embodiments 1 to 18, wherein the adjuvant comprises a TLR4 agonist.

Embodiment 20 is the vaccine combination of Embodiment 19, wherein the adjuvant comprises an oil-in-water emulsion and a TLR4 agonist.

Embodiment 21 is the vaccine combination of Embodiment 19, wherein the adjuvant comprises a liposome with QS21 and a TLR4 agonist.

Embodiment 22 is the vaccine combination of any one of Embodiments 19 to 21, wherein the TLR4 agonist is a lipid A analog or derivative.

Embodiment 23 is the vaccine combination of Embodiment 22, wherein the TLR4 agonist comprises one or more of MPL, 3D-MPL, RC529, GLA, SLA, E6020, PET-lipid A, PHAD, 3D-PHAD, 3D-(6-acyl)-PHAD, ONO4007, or OM-174.

Embodiment 24 is the vaccine combination of any one of Embodiments 1 to 23, wherein the FimH polypeptide is present in a first composition, the one or more conjugates comprising an E. coli O-antigen polysaccharide covalently coupled to a carrier protein are present in a second composition, and the adjuvant is present in a third composition, preferably, the first, second and third compositions are combined shortly before administration.

Embodiment 25 is the vaccine combination of any one of Embodiments 1 to 23, wherein the FimH polypeptide and the one or more conjugates comprising an E. coli O-antigen polysaccharide covalently coupled to a carrier protein are present in a first composition, the adjuvant is present in a second composition, preferably, the first and second compositions are combined shortly before administration.

Embodiment 26 is the vaccine combination of any one of Embodiments 1 to 23, wherein the FimH polypeptide and the adjuvant are present in a first composition, the one or more conjugates comprising an E. coli O-antigen polysaccharide covalently coupled to a carrier protein are present in a second composition, preferably, the first and second compositions are combined shortly before administration.

Embodiment 27 is the vaccine combination of any one of Embodiments 1 to 23, wherein the one or more conjugates comprising an E. coli O-antigen polysaccharide covalently coupled to a carrier protein and the adjuvant are present in a first composition, the FimH polypeptide is present in a second composition, preferably, the first and second compositions are combined shortly before administration.

Embodiment 28 is the vaccine combination of any one of Embodiments 1 to 23, wherein the FimH polypeptide, the one or more conjugates comprising an E. coli O-antigen polysaccharide covalently coupled to a carrier protein, and the adjuvant are present in a single composition.

Embodiment 29 is a method for inducing an immune response against a urinary tract infection caused by E. coli in a subject in need thereof, comprising administering to the subject a vaccine combination of any one of Embodiments 1 to 28.

Embodiment 30 is the method of Embodiment 29, wherein the subject is a human female between about 16 and about 50 years old, e.g. between about 16 and about 35 years old.

Embodiment 31 is the method of Embodiment 29, wherein the subject is a human adult more than 50 years old, or more than 55 years old, or more than 60 years old, or more than 65 years old.

Embodiment 32 is the method of Embodiment 29, wherein the subject is a human subject suffering from reoccurring UTIs.

Embodiment 33 is the method of Embodiment 29, wherein the subject is a human subject having or at risk of acquiring E. coli bacteremia or sepsis.

Embodiment 34 is the method of Embodiment 29, wherein the subject is a human subject that has a condition which requires catheter usage.

Embodiment 35 is the method of Embodiment 29, wherein the subject is a human subject that undergoes a pre-scheduled surgery.

Embodiment 36 is the method of Embodiment 29, wherein the subject is a human subject that has diabetes.

Embodiment 37 is the method of any one of Embodiments 29 to 36, wherein the method prevents or reduces a symptom of urinary tract infection.

Embodiment 38 is a use of a vaccine combination of any one of Embodiments 1 to 28 in the manufacture of a medicament for inducing an immune response to extra-intestinal pathogenic E. coli (ExPEC) in a subject in need thereof.

Embodiment 39 is a use of a vaccine combination of any one of Embodiments 1 to 28 for preventing urinary tract infection (UTI), or for reducing the chance of suffering from or for reducing the severity of one or more symptoms associated with UTI in a subject in need thereof.

Embodiment 40 is a method for making the vaccine combination of any one of Embodiments 1 to 23 or 28, comprising combining the FimH polypeptide, the one or more conjugates comprising an E. coli O-antigen polysaccharide covalently coupled to a carrier protein, and the adjuvant, to obtain the vaccine combination.

Embodiment 41 is the vaccine combination of any one of Embodiments 1-12, wherein the FimH polypeptide comprises SEQ ID NO: 9.

Embodiment 42 is the vaccine combination of any one of Embodiments 1-12, wherein the FimH polypeptide comprises a mutation of arginine to proline at position 60, wherein the amino acids are numbered in alignment with SEQ ID NO: 9.

Embodiment 43 is a method of Embodiment 29, wherein the vaccine combination is administered to the subject by multiple compositions within a time frame and at a location that allows draining of the vaccine combination components to the same lymph node.

### Examples

The following examples of the invention are to further illustrate the nature of the invention. It should be understood that the following examples do not limit the invention and that the scope of the invention is to be determined by the appended claims.

### Example 1: Composition Components

O-Antigen Bioconjugates

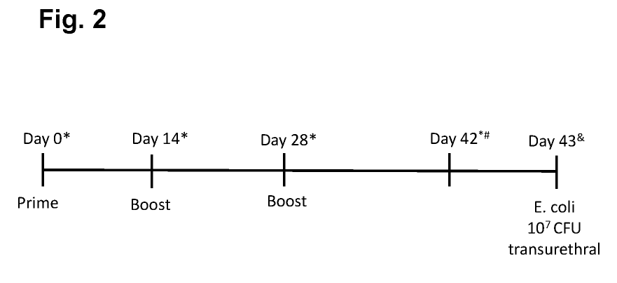

O1A-EPA, O2-EPA, O6A-EPA and O25B-EPA bioconjugates containing, respectively, E. coli O1A, O2, O6A and O25B covalently linked to the glycosylation sites of an EPA protein carrier can be produced, purified, and characterized as described in, e.g., Ihssen et al., 2010, supra, and in WO 2006/119987, WO 2009/104074, and in particular in WO 2015/124769 and WO 2017/035181, the disclosures of which are incorporated by reference herein. The bioconjugates are synthesized using recombinant E. coli cells, which express the polysaccharide-synthesizing enzymes of the different O-serotypes in the presence of oligosaccharyltransferase PglB, and a protein carrier (EPA). In this approach, the glycoconjugate vaccine can be expressed in the periplasm of E. coli, extracted and purified through a biochemical process (for example illustrated in FIGS. 1 and 2 of WO 2017/035181). Table 1 indicates examples of host strains that can be used for the production of conjugates according to an embodiment of the invention.

TABLE 1
Examples of host strains for production of bioconjugates

| | | EPA expression | PglB expression |
|---|---|---|---|
| Product | Strain | plasmid | plasmid |
| EPA-O1A | W3110 Δrfb::rfb(upec032) ΔwaaL | pGVXN1076 | pGVXN970 |
| EPA-O2 | W3110 Δrfb::rfb(upec116) ΔwaaL | pGVXN1076 | pGVXN971 |
| EPA-O6A | W3110 Δrfb::rfb(CCUG11309) ΔwaaL | pGVXN659 | pGVXN114 |
| EPA-O25B | W3110 Δrfb::rfb(upec138) ΔwaaL ΔgtrABS | pGVXN1076 | pGVXN970 |

For example, for O25B-EPA production, a strain with a genomically integrated O25B cluster was constructed, the resulting recombinant host cells were used for production of O25B-EPA bioconjugates in the periplasm, and the O25B-EPA bioconjugate was purified, all as described in WO 2017/035181. Similarly the O1A-EPA, O2-EPA, and O6A-EPA bioconjugates were prepared as described in WO 2017/035181.

Compositions comprising all four bioconjugates O25B-EPA, O1A-EPA, O2-EPA and O6A-EPA were prepared by mixing the four bioconjugates in a ratio of 1:1:1:1 or 2:1:1:1, as described in WO 2017/035181. Such compositions are referred to herein as ExPEC4V, for brevity.

FimH

FimH can be recombinantly expressed by conventional methods for production of recombinant proteins in E. coli. For the experiments herein, FimHt or FimH_{LD} respectively having the sequences provided in SEQ ID NO: 5 and SEQ ID NO: 9 (referred to herein as FimH_{LD} 23-10) is used (these are examples of high affinity FimH variants). In addition, a FimH_{LD} 23-10 sequence with a proline-to-arginine substitution at position 60 (R60P) was also used (this is an example of a low affinity FimH variant, see e.g. Rabbani et al, 2018, J Biol Chem, supra).

Sequences encoding FimHt, FimH_{LD} 23-10, and FimH_{LD} 23-10 (R60P), each preceded by a signal peptide and containing a (cleavable) His-tag are cloned into an expression vector and purified from the periplasm using Ni-affinity purification after osmotic shock, according to methods known in the art (see e.g. Schembri et al, 2000, supra).

Adjuvant

The adjuvants used are include Quil-A® adjuvant (saponin vaccine adjuvant, obtained from Invivogen, catalog # vac-quil) or Alum (aluminum hydroxide, Alhydrogel 2%®, obtained from Invivogen, catalog # vac-alu-250). In additional experiments, TLR4 agonist adjuvant AS01B (suspension with 5 ug 3-O-desacyl-4′-monophosphoryl lipid A (MPL) from Salmonella minnesota and 5 ug QS-21; see e.g. https://www.ema.europa.eu/documents/product-information/shingrix-epar-product-information en.pdf; Didierlaurent A M, et al, 2017, Expert Review of Vaccines, 16:1, 55-63, DOI: 10.1080/14760584.2016.1213632), is used.

Composition

Compositions comprising ExPEC4V, FimH, and/or adjuvant are prepared by mixing each of the individual respective components together before injection. For example, ExPEC4V and FimH may be mixed into an antigen composition, while adjuvant is separate and may be mixed with antigen composition just before administration. Adjuvants used are described in tables in examples below.

### Example 2: Methods

FimH ELISA

96-well plates are coated overnight with 1 ug/mL of FimH. After washing, coated wells are incubated with blocking buffer [phosphate-buffered saline (PBS)+2% bovine serum albumin (BSA)] for 2 hours at room temperature. After washing with PBS+0.05% Tween 20, serum is added to the plates that are then incubated for 1 hour at room temperature. After washing, goat anti-mice antibody conjugated to horseradish peroxidase diluted in PBS with 2% BSA is added to each well for 1 hour at room temperature. After a final washing, the reaction is developed with tetramethylbenzidine substrate. The reaction is stopped with 1M phosphoric acid, and absorbance is measured at 450 nm.

O-Antigen and EPA ELISA

ELISA plates are coated with 2.5 ug/mL of purified O-LPS and 5 ug/mL of methylated bovine serum albumin in PBS or with 1 ug/mL of EPA in PBS. Anti-mouse IgG antibody conjugated with horseradish peroxidase is added to the plates, followed by the substrate tetramethylbenzidine. The reaction is stopped with 1M H2SO4, and absorbance is measured at 450 nm.

Opsonophagocytic Assay (OPA)

Heat-inactivated serum samples are serially diluted in buffer with approximately 10³ CFU/well of the respective E. coli serotype and incubated for 30 min on a shaker. Pre-absorbed human complement (12.5% final concentration) and differentiated HL60 cells are added to the assay plate at a 600:1 cell-to-bacterium ratio. After 16 hr incubation at 33° C., the reaction mixture is spotted onto agar plates and the colonies that grow are enumerated.

Adhesion of Bladder Cells

Bacteria (E. coli J96) are labeled with a fluorescein isothiocyanate (FITC). Labeled bacteria are incubated with bladder urothelial cells (5637 cell line) for 1 h at 37° C. The % of adherent bacteria is measured by flow cytometry. For evaluation of serum inhibition, bacteria are previously incubated with serum samples for 30 minutes at 37° C. and then mixed with 5637 cells.

Antibody-Secreting Cells (ASC) and Memory B Cells Enumeration by ELISpot

Total splenocytes are stimulated for 5 days with de-lipidated O-LPS (2.5 ug/ml), CpG (3 ug/ml) and IL2 (50UI/ml). After incubation, cell suspension is adjusted to 10⁷ cells/mL. ELISpot plates are coated with O-LPS (5 μg/mL) in PBS and incubated overnight at 4° C. After washing (PBS) and blocking for 2 h at room temperature, cell suspension is added to the plates (3×10⁶ cells/well) in triplicate in a 3-fold serial dilution. Plates are incubated at 37° C. for 5 h. After washing, detection antibody HRP-conjugated anti-IgG is added to the plates and incubated overnight at 4° C. The substrate solution is then added to the plates and the reaction is developed in the dark for 10 min; after washing 10-20 times with double-distilled water, plates are dried and the number of spots, corresponding to individual ASCs, are enumerated.

T Cell Proliferation and Cytokine Secretion

Splenocytes are isolated in RPMI 1640 supplemented with 5% fetal calf serum and run through sterile steel mesh to remove large particles. After removal of supernatant and erythrocytes lysis, cell suspension is washed three times in RPMI 1640 supplemented with 5% fetal calf serum and centrifugated at 1000 rpm. Cell suspension is adjusted to 2×10⁶ cells/mL and stimulated in vitro with 5 and 10 ug/ml of FimH or EPA for 24, 48 and 72 hr at 37° C., 5% CO₂. Non-stimulated splenocytes are cultured under the same conditions and used as negative control; cells stimulated with anti-CD3/CD28 are used as positive control. At the beginning of the antigen-specific stimulation, cells are labeled with CFSE and at each time-point (24, 48 and 72 hr), cells are harvested and stained with monoclonal antibodies anti-CD3, CD4, CD8, IFNg, TNFa, IL10, IL4 and IL2. In addition, the levels of cytokines (IFNg and IL5) secreted in culture supernatant of splenocytes stimulated in vitro with FimH or EPA (5 and 10 ug/mL) for 72 h is determined by ELISA.

### Example 3: Initial Experiments with O-Conjugates+ FimH in Animals

Preliminary experiments were set up in C3H/HeN mice (using intramuscular (i.m.) immunizations with doses of FimH (25 ug/dose) administered at day 0 (prime) and day 28 (boost) alone or in combination with adjuvant QuilA (15 ug/dose), or with ExPEC4V containing 8, 4, 8 and 16 ug of O1A, O2, O6A and O25B polysaccharides/dose, respectively, administered at day 0 (prime), day 14 (boost 1) and day 28 (boost 2) alone or in combination with QuilA, or combinations of ExPEC4V and FimH without or with QuilA adjuvant [or with the comparator adjuvant Alhydrogel (aluminum hydroxide, 150 ug/dose)]; in certain experiments, serum antibody levels induced by the different formulations of the vaccine were evaluated at day 0 (pre-vaccination), day 14, 28 and 42 (post-vaccination); in certain experiments, FimH and carrier (EPA)-mediated T cell responses and memory B cells were evaluated using total splenocytes harvest at day 42 post-immunization, and in certain experiments, the functionality of serum antibodies was evaluated by OPA and by antibody-mediated inhibition of adhesion/invasion of bladder cells at day 42 post-vaccination).

Finding an optimal dosage for each of the components in this mouse strain, in particular for the ExPEC4V components, appeared not straightforward. However, some conclusions could be drawn from these initial studies. The preliminary results in C3H/HeN mice did show that all formulations tested effectively induced production of antigen-specific antibodies and the magnitude of the antibody response was significantly increased when the formulations were combined with an adjuvant. In agreement therewith, the number of antibody-secreting cells (ASC) was also significantly increased when the formulations were administered in combination with an adjuvant. Notably, the adjuvanted formulations induced a predominant secretion of IFNg by splenocytes re-stimulated in vitro with EPA or FimH. These preliminary findings suggest that the adjuvanted formulations predominantly activate Th1 effector T cell responses.

Though these experiments showed encouraging results, additional studies are needed to further optimize the dose of each vaccine component and adjuvant for this mouse strain. Instead of using this mouse model, the use of a different pre-clinical model can corroborate the data obtained in mice and can bring a better understanding of the vaccine-induced immune response. The experiments performed in a second pre-clinical model, namely Sprague Dawley rats, are described below in Example 4 (Table 2 and FIG. 2).

Initial experiments performed in Sprague Dawley rats showed that ExPEC4V induced high levels of O-antigen-specific antibodies against all vaccine-related serotypes (e.g. van den Dobbelsteen, Vaccine, 34: 4152-4160, 2016). Importantly, the functionality of vaccine-induced antibodies was demonstrated by their ability to mediate bacterial opsonophagocytic killing (Table 2). Sprague Dawley rats received 3 intramuscular immunizations with ExPEC4V containing 4 or 0.4 ug of each O1A, O2, O6A and O25B polysaccharides/dose at day 0, 14 and 28. Evaluation of opsonic antibodies showed that rats immunized with 0.4 μg of ExPEC4V had on average higher opsonic titers against O2 and O25B E. coli strains compared to animals immunized with 4 μg/dose (Table 2). The opsonic titers observed against O6A E. coli strain were similar at either vaccine dose tested (Table 2). In summary, ExPEC4V was immunogenic in rats; high levels of O-antigen-specific antibodies were detected post-vaccination and, importantly, these antibodies were functional, capable of mediating opsonophagocytic killing of E. coli.

At the time these studies were performed, the OPA assay was developed only for three E. coli strains (O2, O6A and O25B). Therefore, the functionality of antibodies induced by O1A conjugate were not described in Table 2. However, when the assay was further developed and qualified for use with human serum samples, functionality was demonstrated against all serotypes included in the vaccine (O1A, O2, O6A and O25B, not shown).

TABLE 2
Functionality of antibodies induced by ExPEC4V in rats.

| | Strains | | | | | | | | | | | |
|---|---|---|---|---|---|---|---|---|---|---|---|---|
| | ExPEC4V | | | | | | | | | | | |
| | μg each | O2 | | | O6A | | | | O25B | | | |
| | PS/dose | 0.4 | | 4.0 | 0.4 | | 4.0 | | 0.4 | | 4.0 | |
| Animals | Experiments | 1 | 2 | 1 | 1 | 2 | 1 | 2 | 1 | 2 | 1 | 2 |
| 1 | Pre | 6 | 7 | 5 | 17 | 6 | 6 | 16 | 2404 | 2082 | 0 | 0 |
| | Post | >16384 | 1476 | 293 | 202 | 226 | 2045 | 2821 | 1847 | 1578 | 9 | 0 |
| 2 | Pre | 21 | 11 | 11 | 11 | 90 | 0 | 0 | 0 | 0 | 0 | 0 |
| | Post | 11148 | >16384 | 150 | 436 | 475 | 10262 | 11460 | 0 | 0 | 4 | 0 |
| 3 | Pre | 6 | 6 | 0 | 0 | 5 | 0 | 0 | 0 | 0 | 0 | 0 |
| | Post | 11073 | >16384 | 46 | 98 | 37 | 7959 | 8597 | 6 | 0 | 355 | 197 |
| 4 | Pre | 5 | 5 | 5 | 23 | 17 | 0 | 0 | 0 | 0 | 0 | 0 |
| | Post | >16384 | 63 | 57 | 108 | 116 | 2189 | 4488 | 0 | 0 | 70 | 26 |
| 5 | Pre | 7 | 0 | 0 | 30 | 8 | 8 | 7 | 0 | 0 | 0 | 0 |
| | Post | 10413 | 7050 | 105 | >16384 | 12672 | 3107 | 7564 | 0 | 0 | 105 | 69 |
| 6 | Pre | 8 | 0 | 8 | 299 | 164 | 5 | 0 | 269 | 154 | 0 | 0 |
| | Post | 89 | 34 | 24 | 1725 | 1475 | 540 | 896 | 0 | 0 | 0 | 0 |
| 7 | Pre | 9 | 9 | 6 | 18 | 21 | 22 | 5 | 3 | 0 | 0 | 0 |
| | Post | >16384 | >16384 | 109 | 1249 | 1863 | 160 | 143 | 1130 | 630 | 9 | 8 |
| 8 | Pre | 4 | 6 | 6 | 26 | 22 | 0 | 0 | 0 | 0 | 0 | 0 |
| | Post | 5058 | 4201 | 39 | 6590 | 3826 | 288 | 656 | 3336 | 1986 | 0 | 0 |
| Average | Pre | 8 | 5 | 5 | 53 | 42 | 5 | 3 | 334 | 280 | 0 | 0 |
| | Post | 10867 | 7747 | 103 | 3349 | 2586 | 3319 | 4578 | 790 | 524 | 69 | 37 |

The results obtained with this pre-clinical rat model are in line with findings for this vaccine in humans (Phase 1b clinical trial), where ExPEC4V induced a robust immune response and antibody functionality was demonstrated for all vaccine-related serotypes (e.g. Huttner A, et al., 2017, supra).

Further initial experiments in rats showed that FimH immunization induced antibodies that inhibit bacterial adhesion to bladder epithelial cells. Immunizations of Wistar rats with 4 intramuscular doses of 2 different variants of FimH (FimH_{LD} 23-10 and FimH_{LD} 23-10 (R60P); 60 ug each variant/dose combined with a non-Freund adjuvant as part of Speedy 28-Day model, Eurogentec, secure.eurogentec.com/speedy.htlm) induced functional antibodies that are able to reduce bacterial adhesion to bladder epithelial cells (FIG. 1).

It is therefore believed that a combination of E. coli O-conjugates and FimH in the presence of adjuvant result in an improved vaccine against UTI, by combining the different modes of action, as compared to vaccines based on either O-antigens or adjuvanted FimH alone.

### Example 4. Immunogenicity and Efficacy of O-Conjugates and FimH in Rats

Sprague Dawley rats (females, 6-7 weeks) receive 3 intramuscular immunizations of FimH (60 ug/dose) administered at day 0, 14 and 28 alone or in combination with the adjuvant AS01B (Group 1 and 2, Table 3, FIG. 2). Group 3 and 4 receive 3 doses of ExPEC4V containing 0.4 ug of each polysaccharide (O1A, O2, O6B and O25B) administered at day 0, 14 and 28 alone or in combination with AS01_{B} (Group 3 and 4, Table 3, FIG. 2). Group 5 and 6 receive the combined formulation, containing FimH (60 ug/dose) and ExPEC4V (0.4 ug of each polysaccharide) with or without adjuvant (Group 5 and 6, Table 3, FIG. 2). The adjuvant AS01B, is administered at 5 ug MPL and 5 ug QS21 (i.e. 1/10 of a human dose) (Table 3). As control groups, the animals are immunized only with the adjuvant AS01B (Group 7) or saline (Group 8).

Serum antibody levels induced by the different formulations of the vaccine are evaluated at day 0 (pre-vaccination) and day 14, 28 and 42 (post-vaccination). FimH and carrier (EPA)-mediated T cell responses and memory B cells are evaluated using total splenocytes harvest at day 42 post-immunization. In addition, the functionality of serum antibodies is evaluated by OPA and by antibody-mediated inhibition of adhesion of bladder cells at day 42 post-vaccination.

At day 43 post-immunization, the animals are challenged with 10⁷ CFU of E. coli via transurethral catheterization. Bladder and kidney CFU are determined 4 h and 6 days post-challenge.

TABLE 3
Immunogenicity and efficacy study in Sprague Dawley rats.

| Groups | Prime (d0) | Boost (d14) | Boost (d28) |
|---|---|---|---|
| 1 | FimH | FimH | FimH |
| 2 | FimH + AS01_{B} | FimH + AS01_{B} | FimH + AS01_{B} |
| 3 | ExPEC4V | ExPEC4V | ExPEC4V |
| 4 | ExPEC4V + AS01_{B} | ExPEC4V + AS01_{B} | ExPEC4V + AS01_{B} |
| 5 | FimH + ExPEC4V | FimH + ExPEC4V | FimH + ExPEC4V |
| 6 | FimH + | FimH + | FimH + |
| | ExPEC4V + AS01_{B} | ExPEC4V + AS01_{B} | ExPEC4V + AS01_{B} |
| 7 | AS01_{B} | AS01_{B} | AS01_{B} |
| 8 | Saline | Saline | Saline |

FimH: 60 ug/dose;
AS01_{B}: 5 ug MPL and 5 ug QS21 per dose;
ExPEC4V containing 0.4 ug of each polysaccharide (O1A, O2, O6A and O25B) per dose.

Sequences

| Description | SEQUENCE | SEQ ID NO. |
|---|---|---|
| Detoxified EPA | GSGGGDQNATGSGGGKLAEEAFDLWNECAKACVLDLKDG | 1 |
| protein comprising | VRSSRMSVDPAIADTNGQGVLHYSMVLEGGNDALKLAID | |
| 4 optimized N- | NALSITSDGLTIRLEGGVEPNKPVRYSYTRQARGSWSLN | |
| glycosylation | WLVPIGHEKPSNIKVFIHELNAGNQLSHMSPIYTIEMGD | |
| sequences | ELLAKLARDATFFVRAHESNEMQPTLAISHAGVSVVMAQ | |
| | AQPRREKRWSEWASGKVLCLLDPLDGVYNYLAQQRCNLD | |
| | DTWEGKIYRVLAGNPAKHDLDIKDNNNSTPTVISHRLHF | |
| | PEGGSLAALTAHQACHLPLEAFTRHRQPRGWEQLEQCGY | |
| | PVQRLVALYLAARLSWNQVDQVIRNALASPGSGGDLGEA | |
| | IREQPEQARLALTLAAAESERFVRQGTGNDEAGAASADV | |
| | VSLTCPVAKDQNRTKGECAGPADSGDALLERNYPTGAEF | |
| | LGDGGDVSFSTRGTQNWTVERLLQAHRQLEERGYVFVGY | |
| | HGTFLEAAQSIVFGGVRARSQDLDAIWRGFYIAGDPALA | |
| | YGYAQDQEPDARGRIRNGALLRVYVPRWSLPGFYRTGLT | |
| | LAAPEAAGEVERLIGHPLPLRLDAITGPEEEGGRVTILG | |
| | WPLAERTVVIPSAIPTDPRNVGGDLDPSSIPDKEQAISA | |
| | LPDYASQPGKPPREDLKLGSGGGDQNAT | |
| N-glycosylation | Asp(Glu)-X-Asn-Z-Ser(Thr), wherein X | 2 |
| consensus sequence | and Z are independently selected from | |
| | any natural amino acid except Pro | |
| N-glycosylation | Asn-X-Ser(Thr), wherein X can be any | 3 |
| consensus sequence | amino acid except Pro | |
| Example of FimH | MKRVITLFAVLLMGWSVNAWSFACKTANGTAIPIGGGSA | 4 |
| (full length) | NVYVNLAPVVNVGQNLVVDLSTQIFCHNDYPETITDYVT | |
| sequence | LQRGSAYGGVLSNFSGTVKYSGSSYPFPTTSETPRVVYN | |
| | SRTDKPWPVALYLTPVSSAGGVAIKAGSLIAVLILRQTN | |
| | NYNSDDFQFVWNIYANNDVVVPTGGCDVSARDVTVTLPD | |
| | YPGSVPIPLTVYCAKSQNLGYYLSGTTADAGNSIFTNTA | |
| | SFSPAQGVGVQLTRNGTIIPANNTVSLGAVGTSAVSLGL | |
| | TANYARTGGQVTAGNVQSIIGVTFVYQ | |
| Example of a | FACKTANGTAIPIGGGSANVYVNLAPVVNVGQNLVVDLS | 5 |
| FimHt sequence | TQIFCHNDYPETITDYVTLQRGSAYGGVLSNFSGTVKYS | |
| | GSSYPFPTTSETPRVVYNSRTDKPWPVALYLTPVSSAGG | |
| | LVIKAGSLIAVLILRQTNNYNSDDFQFVWNIYANNDVVV | |
| | PTGGCDVSARDVTVTLPDYRGSVPIPLTVY | |
| SEQ ID NO: 2 of | MKRVITLFAVLLMGWSVNAWSFACKTANGTAIPIGGGSA | 6 |
| U.S. Pat. No. | NVYVNLAPVVNVGQNLVVDLSTQIFCHNDYPETITDYVT | |
| 6,500,434 | LQRGSAYGGVLSNFSGTVKYSGSSYPFPTTSETPRVVYN | |
| (example full | SRTDKPWPVALYLTPVSSAGGVAIKAGSLIAVLILRQTN | |
| length FimH | NYNSDDFQFVWNIYANNDVVVPTGGCDVSARDVTVTLPD | |
| sequence) | YRGSVPIPLTVYCAKSQNLGYYLSGTHADAGNSIFTNTA | |
| | SFSPAQGVGVQLTRNGTIIPANNTVSLGAVGTSAVSLGL | |
| | TANYARTGGQVTAGNVQSIIGVTFVTQ | |
| SEQ ID NO: 29 of | FACKTANGTAIPIGGGSANVYVNLAPVVNVGQNLVVDLS | 7 |
| U.S. Pat. No. | TQIFCHNDYPETITDYVTLQRGSAYGGVLSNFSGTVKYS | |
| 6,737,063 | GSSYPFPTTSETPRVVYNSRTDKPWPVALYLTPVSSAGG | |
| (example FimH | LVIKAGSLIAVLILRQTNNYNSDDFQFVWNIYANNDVVV | |
| sequence with | PTGGCDVSARDVTVTLPDYRGSVPIPLTVYCAKSQNLGY | |
| truncation at N- | YLSGTHADAGNSIFTNTASFSPAQGVGVQLTRNGTIIPT | |
| terminus) | NNTVSLGAVGTSAVSLGLTANYARTGGQVTAGNVQSIIG | |
| | VTFVYQ | |
| Example of a | FACKTANGTAIPIGGGSANVYVNLAPVVNVGQNLVVDLS | 8 |
| FimH_{LD} sequence | TQIFCHNDYPETITDYVTLQRGSAYGGVLSNFSGTVKYS | |
| | GSSYPFPTTSETPRVVYNSRTDKPWPVALYLTPVSSAGG | |
| | LVIKAGSLIAVLILRQTNNYNSDDFQFVWNIYANNDVVV | |
| | PTGG | |
| Example of a | FACKTANGTAIPIGGGSANVYVNLAPAVNVGQNLVVDLS | 9 |
| FimH_{LD} sequence | TQIFCHNDYPETITDYVTLQRGSAYGGVLSNFSGTVKYS | |
| (FimH_{LD} 23-10) | GSSYPFPTTSETPRVVYNSRTDKPWPVALYLTPVSSAGG | |
| | VAIKAGSLIAVLILRQTNNYNSDDFQFVWNIYANNDVVV | |
| | PTG | |
| Example of FimH | MKRVITLFAVLLMGWSVNAWSFACKTANGTAIPIGGGSA | 10 |
| (23-10) | NVYVNLAPAVNVGQNLVVDLSTQIFCHNDYPETITDYVT | |
| (full length) | LQRGSAYGGVLSNFSGTVKYSGSSYPFPTTSETPRVVYN | |
| sequence | SRTDKPWPVALYLTPVSSAGGVAIKAGSLIAVLILRQTN | |
| | NYNSDDFQFVWNIYANNDVVVPTGGCDVSARDVTVTLPD | |
| | YPGSVPIPLTVYCAKSQNLGYYLSGTTADAGNSIFTNTA | |
| | SFSPAQGVGVQLTRNGTIIPANNTVSLGAVGTSAVSLGL | |
| | TANYARTGGQVTAGNVQSIIGVTFVYQ | |
| Example FimH (23- | FACKTANGTAIPIGGGSANVYVNLAPAVNVGQNLVVDLS | 11 |
| 10) sequence with | TQIFCHNDYPETITDYVTLQRGSAYGGVLSNFSGTVKYS | |
| truncation at N- | GSSYPFPTTSETPRVVYNSRTDKPWPVALYLTPVSSAGG | |
| terminus | VAIKAGSLIAVLILRQTNNYNSDDFQFVWNIYANNDVVV | |
| | PTGGCDVSARDVTVTLPDYPGSVPIPLTVYCAKSQNLGY | |
| | YLSGTTADAGNSIFTNTASFSPAQGVGVQLTRNGTIIPA | |
| | NNTVSLGAVGTSAVSLGLTANYARTGGQVTAGNVQSIIG | |
| | VTFVYQ | |

The embodiments of the invention are intended to be merely exemplary, and those skilled in the art will recognize, or be able to ascertain using no more than routine experimentation, numerous equivalents to the specific procedures of the invention. All such equivalents are considered to be within the scope of the present invention and are covered by the following claims.

All references (including patent applications, patents, and publications) cited herein are incorporated herein by reference in their entirety and for all purposes to the same extent as if each individual publication or patent or patent application was specifically and individually indicated to be incorporated by reference in its entirety for all purposes.

### REFERENCES

1. Brumbaugh A R and Mobley H L T, 2012, Expert Rev Vaccines, 11: 663-676
2. Huttner A, et al., 2017, Lancet Infect Dis, dx.doi.org/10.1016/S1473-3099(17)30108-1
3. Langermann S, et al., 1997, Science, 276: 607-611
4. Langermann S, et al., 2000, J Infect Dis, 181: 774-778
5. O'Brien V P et al., 2016, Nat Microbiol, 2:16196
6. Russo et al., 2003, Microbes Infect, 5(5):449-56
7. Boudeau J et al, 1999, Infect Immun 67: 4499-4509
8. Nash J H E et al, 2010, BMC Genomics 11: 667
9. Conte M P et al, 2014, BMC Research Notes 7: 748
10. Desilets M et al, 2016, Inflamm Bowel Dis 22: 1-12
11. Martinez-Medina M and LJ Garcia-Gil, 2014, World J Gastrointest Pathophysiol. 15: 213-227
12. WO 2017/035181
13. WO 2015/124769
14. Stenutz et al., 2006, FEMS Microbial Rev, 30: 382-403
15. Cryz et al, 1995, Vaccine 13: 449-453
16. WO 2006/119987
17. WO 2009/104074
18. Ihssen et al., 2010, Microbial Cell Factories, 9:61
19. Apicella et al., 1994, Methods Enzymol, 235:242-52
20. Woodward et al., 2010, Nat Chem Biol, 6(6): 418-423
21. Lukac et al., 1988, Infect Immun, 56: 3095-3098
22. Ho et al., 2006, Hum Vaccin, 2:89-98
23. Pawlowski et al., 2000, Vaccine, 18:1873-1885
24. Robbins et al., 2009, Proc Natl Acad Sci USA, 106:7974-7978
25. U.S. Pat. No. 5,370,872
26. Fattom A et al, 1999, Vaccine 17: 126-133
27. Micoli F et al, 2013, Anal Biochem 434: 136-145
28. Stefanetti G et al, 2014, Vaccine 32: 6122-6129
29. Stefanetti G et. al., 2015, Angew. Chem. Int. Ed. 54, http://dx.doi.org/10.1002/anie.201506112
30. Stefanetti G et. al., 2015, Bioconjug Chem 26: 2507-2513
31. Meloni E et al, 2015, J Biotechnol 198: 46-52
32. Rondini S et al, 2015, Infect Immun 83: 996-1007
33. Baliban S M et al, 2017, PLoS Neglected Tropical Diseases, doi.org/10.1371/journal.pntd.0005493
34. Saraswat et al., 2013, Biomed. Res. Int., ID#312709 (p. 1-18)
35. U.S. Pat. No. 6,500,434
36. U.S. Pat. No. 6,737,063
37. Choudhury D et al, 1999, Science 285: 1061-1066
38. Sauer M M et al., 2016, Nat Commun, 7:10738
39. WO 2002/004496
40. Rabbani S et al, 2010, Anal Biochem 407: 188-195
41. US20030138449
42. Masson J D et al, 2017, Expert Rev Vaccines 16: 289-299
43. GB2220211
44. EP0971739
45. EP1194166
46. U.S. Pat. No. 6,491,919
47. EP1126876
48. U.S. Pat. No. 7,357,936
49. EP0671948
50. EP0761231
51. U.S. Pat. No. 5,750,110
52. WO2007/109812
53. WO2007/109813
54. Petrovsky N and PD Cooper, 2015, Vaccine 33: 5920-5926
55. US20150056224
56. U.S. Pat. No. 6,676,958
57. Zhu D and W Tuo, 2016, Nat Prod Chem Res 3: e113 (doi:10.4172/2329-6836.1000e113
58. Kensil et al., in Vaccine Design: The Subunit and Adjuvant Approach (eds. Powell & Newman, Plenum Press, N Y, 1995)
59. U.S. Pat. No. 5,057,540
60. EP0399843
61. U.S. Pat. No. 6,299,884
62. U.S. Pat. No. 6,451,325
63. Stoute et al., 1997, N. Engl. J. Med. 336, 86-91
64. US 2011/0206758
65. Bioworld Today, Nov. 15, 1998
66. Reed G, et al., 2013, Nature Med, 19: 1597-1608.
67. Raetz, 1993, J. Bacteriology 175:5745-5753
68. U.S. Pat. No. 5,593,969
69. U.S. Pat. No. 5,191,072
70. EP1385541
71. US20100310602
72. U.S. Pat. No. 8,722,064
73. Carter D et al, 2016, Clin Transl Immunology 5: e108 (doi: 10.1038/cti.2016.63)
74. WO 2013/119856
75. WO 2006/116423
76. U.S. Pat. No. 4,987,237
77. U.S. Pat. No. 4,436,727
78. U.S. Pat. No. 4,877,611
79. U.S. Pat. No. 4,866,034
80. U.S. Pat. No. 4,912,094
81. U.S. Pat. No. 6,759,241
82. U.S. Pat. No. 9,017,698
83. U.S. Pat. No. 9,149,521
84. U.S. Pat. No. 9,149,522
85. U.S. Pat. No. 9,415,097
86. U.S. Pat. No. 9,415,101
87. U.S. Pat. No. 9,504,743
88. Johnson et al., 1999, J Med Chem, 42:4640-4649
89. Ulrich and Myers, 1995, Vaccine Design: The Subunit and Adjuvant Approach
90. Powell and Newman, Eds.; Plenum: New York, 495-524
91. “Remington's pharmaceutical sciences,” XIII ed. Editor-in-Chief Eric W. Martin. Mack Publishing Co., Easton, Pa., 1965
92. Raetz C R and C Whitfield, 2002, Annu Rev Biochem, 71: 635-700
93. Ireton G C and SG Reed, 2013, Expert Review of Vaccines, 12: 793-807
94. Reed S G et al., 2016, Current Opin Immunol, 41: 85-90
95. http://www.avantilipids.com/divisions/adjuvants
96. Alving C R et al., 2012, Curr Opin Immunol, 24: 310-315
97. Barnhart M M et al, 2000, Proc Natl Acad Sci USA, 97: 7709-7714
98. Barnhart M M et al, 2003, J Bacteriol. 185: 2723-2730
99. Schembri et al., 2000, FEMS Microbiol Letters 188: 147-51
100. Schwartz et al., 2013, Proc Natl Acad Sci USA 110: 15530-15537
101. Gregg K A et al., 2017, MBio 8: 00492-17. doi: 10.1128/mBio.00492-17
102. Didierlaurent A M et al, 2017, Expert Review of Vaccines 16(1): 55-63
103. Le Trong et al, 2010, Cell 141: 645-655
Kalas et al, Sci Adv. 2017, Feb. 10; 3(2):e1601944, doi: 10.1126/sciadv.1601944. eCollection 2017 February, PMID: 28246638
105. Kisiela et al, 2013, Proc Natl Acad Sci USA 110: 19089-19094
106. Rabbani et al, 2018, J Biol Chem 293: 1835-1849

1. A vaccine combination comprising a FimH polypeptide, one or more conjugates comprising an E. coli O-antigen polysaccharide covalently coupled to a carrier protein, and an adjuvant.

2. The vaccine combination of claim 1, wherein the one or more conjugates comprise E. coli O25B antigen polysaccharide.

3. The vaccine combination of claim 2, wherein the conjugates further comprise E. coli O1A antigen polysaccharide, E. coli O2 antigen polysaccharide, and E. coli O6A antigen polysaccharide.

4. The vaccine combination of claim 3, wherein the conjugates further comprise E. coli O-antigen polysaccharide from one or more of O4, O7, O9, O11, O12, O22, O75, O8, O15, O16, or O18 antigen polysaccharides.

5. The vaccine combination of claim 1, wherein the carrier protein is detoxified exotoxin A of Pseudomonas aeruginosa (EPA).

6. The vaccine combination of claim 1, wherein the FimH polypeptide comprises a truncated form of FimH.

7. The vaccine combination of claim 1, wherein the FimH polypeptide is complexed with FimC (FimCH).

8. The vaccine combination of claim 1, wherein the FimH polypeptide is in the low affinity conformation, for instance by a mutation of arginine to proline at amino acid position 60 (R60P), wherein the amino acid numbering is in alignment with the FimH sequence of SEQ ID NO: 9.

9. The vaccine combination of claim 1, wherein the adjuvant comprises saponins.

10. The vaccine combination of claim 1, wherein the adjuvant comprises a TLR4 agonist.

11. The vaccine combination of claim 10, wherein the TLR4 agonist is lipid A or an analog or derivative thereof.

12. The vaccine combination of claim 1, wherein the FimH polypeptide, the one or more conjugates comprising an E. coli O-antigen polysaccharide covalently coupled to a carrier protein and the adjuvant are present in a single composition.

13. The vaccine combination of claim 1, wherein:
a) the FimH polypeptide and the one or more conjugates comprising an E. coli O-antigen polysaccharide covalently coupled to a carrier protein are present in a first composition, and the adjuvant is present in a second composition; or
b) the FimH polypeptide and the adjuvant are present in a first composition, and the one or more conjugates comprising an E. coli O-antigen polysaccharide covalently coupled to a carrier protein are present in a second composition; or
c) the one or more conjugates comprising an E. coli O-antigen polysaccharide covalently coupled to a carrier protein and the adjuvant are present in a first composition, and the FimH polypeptide is present in a second composition; or
d) the FimH polypeptide is present in a first composition, the one or more conjugates comprising an E. coli O-antigen polysaccharide covalently coupled to a carrier protein are present in a second composition, and the adjuvant is present in a third composition.

14. A method for inducing an immune response against a urinary tract infection caused by E. coli in a subject in need thereof, comprising administering to the subject a vaccine combination of claim 1.

15. The method of claim 14, wherein the subject is one or more of:
(i) a human female between about 16 and about 50 years old, e.g. between about 16 and about 35 years old;
(ii) a human adult more than 50 years old, or more than 55 years old, or more than 60 years old, or more than 65 years old;
(iii) a human subject suffering from reoccurring UTIs;
(iv) a human subject having or at risk of acquiring E. coli bacteremia or sepsis;
(v) a human subject that has a condition which requires catheter usage;
(vi) a human subject that undergoes a pre-scheduled surgery; or
(vii) a human subject that has diabetes.

16. A method for inducing an immune response against a urinary tract infection caused by E. coli in a subject in need thereof, comprising administering to the subject a vaccine combination according to claim 13, wherein the first and second composition, or the first, second and third composition, are administered within a time frame and at a location that allows draining of the vaccine combination components to the same lymph node.

17. A method for making the vaccine combination of claim 1, comprising combining the FimH polypeptide, the one or more conjugates comprising an E. coli O-antigen polysaccharide covalently coupled to a carrier protein, and the adjuvant, to obtain the vaccine combination.

18.-23. (canceled)

24. The vaccine combination of claim 9, wherein the adjuvant comprises QS21.

25. The vaccine combination of claim 11, wherein the TLR4 agonist comprises MPL, 3D-MPL, RC529, GLA, SLA, E6020, PET-lipid A, PHAD, 3D-PHAD, 3D-(6-acyl)-PHAD, ONO4007, or OM-174.

26. The method of claim 17, wherein the vaccine combination is obtained by mixing the components into a single mixed composition.
